(19) United States
(12) Patent Application Publication
(10) Pub. No.: US 2004/0077881 A1
(43) Pub. Date: Apr. 22, 2004

(54) $G(d)1-pyrrolines used in pest control

(76) Inventors: Andrew Plant, Berkshire (GB); Thomas Seitz, Langenfeld (GB); Christoph Erdelen, Leichlingen (DE); Andreas Turberg, Haan (DE); Olaf Hansen, Leichlingen (DE)

Correspondence Address: Bayer Cropscience, Patent Department, 100 Bayer Road, Pittsburgh, PA, 15205-9741, US

(21) Appl. No.: 10/467,992

(22) PCT Filed: Jan. 31, 2002
(86) PCT No.: PCT/EP02/00990

(30) Foreign Application Priority Data
Feb. 13, 2001 (DE) 101 06 455.1

(51) Int. Cl. C07D 207/04

(52) U.S. Cl. 548/577

(57) ABSTRACT

Novel Δ¹-pyrrolines of the formula (I)

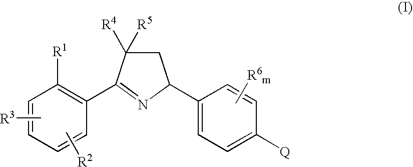

in which

R¹, R², R³, R⁴, R⁵, R⁶, m and Q have the meanings given in the description, a plurality of processes for preparing these substances and their use for controlling. pests.

The present invention relates to novel Δ¹-pyrrolines which are substituted in the 3-position, to a plurality of processes for their preparation and to their use as pesticides.

It is already known that numerous Δ¹-pyrrolines have insecticidal properties (cf. WO 00/21958, WO 99/59968, WO 99/59967 and WO 98/22438). The activity of these substances is good; however, in some cases it is unsatisfactory.

This invention now provides novel Δ¹-pyrrolines of the formula (I)

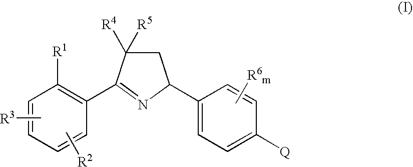

in which

R¹ represents halogen, represents in each case optionally substituted alkyl or alkoxy or represents —S(O)_{w}R⁷,

R² and R³ independently of one another represent hydrogen, halogen or represent in each case optionally substituted alkyl or alkoxy,

R⁴ represents halogen or optionally substituted alkyl or represents —SO₃R⁸,

R⁵ represents hydrogen, halogen or optionally substituted alkyl or represents —SO₃R⁸,

R⁴ and R⁵ furthermore together represent the grouping

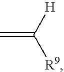

R⁶ represents halogen, represents in each case optionally substituted alkyl, alkoxy or alkylthio,

m represents 0, 1, 2, 3 or 4,

Q represents the grouping —X—Y-Z-E, with the proviso that Y does not represent a direct bond if X does not represent a direct bond,

R⁷ represents optionally substituted alkyl,

R⁸ represents hydrogen or optionally substituted alkyl,

R⁹ represents hydrogen or in each case optionally substituted alkyl, aryl or heteroaryl,

X represents a direct bond, oxygen, —S(O)_{w}—, —NR¹⁰—, carbonyl, carbonyloxy, oxycarbonyl, oxysulphonyl (OSO₂), alkylene, halogenoalkylene, alkenylene, halogenoalkenylene, alkinylene, alkylenoxy, oxyalkylene, oxyalkylenoxy, —S(O)_{w}-alkylene, cyclopropylene or oxiranylene,

Y represents a direct bond or represents in each case optionally substituted phenylene, naphthylene, tetrahydronaphthylene or heterocyclylene,

Z represents a direct bond or —(CH₂)ₙ—,

E represents hydrogen, halogen, hydroxyl, cyano, formyl, nitro, trialkylsilyl, pentafluorothio, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —C(R¹⁸)═N—OR¹⁹, —SO₂NR²⁰OR²¹; or represents in each case optionally substituted alkyl, alkenyl, alkinyl, alkoxy, alkenyloxy, cycloalkyl, cycloalkylalkyl, cycloalkyloxy, aryl, arylalkyl, aryloxy, aryloxyalkyl, saturated or unsaturated heterocyclyl or heterocyclylalkyl,

represents in each case optionally substituted alkyl, cycloalkyl, cycloalkylalkyl, aryl or arylalkyl,

R¹¹ represents in each case optionally substituted alkyl, cycloalkyl, aryl or arylalkyl,

R¹² and R¹³ independently of one another represent hydrogen or —SO₂R¹¹, represents in each case optionally substituted alkyl, alkenyl, cycloalkyl, cycloalkylalkyl, aryl, arylalkyl or saturated or unsaturated heterocyclyl or heterocyclylalkyl,

R¹² and R¹³ furthermore together represent in each case optionally substituted alkenylene or alkylene, where the alkylene chain may in each case be interrupted by —O—, —S— or —NR²²—,

R¹⁴ and R¹⁵ independently of one another represent hydrogen or —SO₂R¹¹, represent in each case optionally substituted alkyl, alkenyl, cycloalkyl, cycloalkylalkyl, aryl, arylalkyl or saturated or unsaturated heterocyclyl or heterocyclylalkyl,

R¹⁴ and R¹⁵ furthermore together represent optionally substituted alkylene, where the alkylene chain may be interrupted by —O—, —S— or —NR²²—,

R¹⁶ and R¹⁷ independently of one another represent hydrogen, represent in each case optionally substituted alkyl, cycloalkyl, cycloalkylalkyl, aryl or arylalkyl,

R¹⁶ and R¹⁷ furthermore together represent in each case optionally substituted alkylene or alkenylene,

R¹⁸ and R¹⁹ independently of one another represent hydrogen, represent in each case optionally substituted alkyl or alkenyl,

R²⁰ and R²¹ independently of one another represent hydrogen, represent in each case optionally substituted alkyl or cycloalkyl,

R²⁰ and R²¹ furthermore together represent in each case optionally substituted alkylene, alkoxyalkylene or alkylthioalkylene,

R²² represents hydrogen, —SO₂R¹¹, —COR¹¹ or —CO₂R¹¹; represents in each case optionally substituted alkyl, alkenyl, cycloalkyl, aryl, or saturated or unsaturated heterocyclyl,

w represents 0, 1 or 2,

n represents 1, 2, 3 or 4.

Depending on the type and number of substituents, the compounds of the formula (I) may be present as geometrical and/or optical isomers, regioisomers or configurational isomers or isomer mixtures thereof of varying composition. What is claimed by the invention are both the pure isomers and the isomer mixtures.

Furthermore, it has been found that the novel compounds of the formula (I) can be obtained by one of the processes described below.

A) Δ¹-Pyrrolines of the formula (I-a)

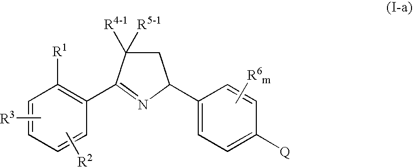

in which

R¹, R², R³, R⁶, m and Q have the meanings given above,

R⁴⁻¹ represents chlorine or bromine,

R⁵⁻¹ represents hydrogen, chlorine or bromine, are obtained by reacting

A1) Δ¹-pyrrolines of the formula (II)

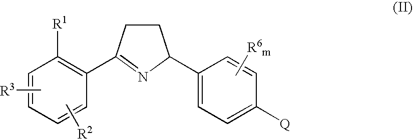

in which

R¹, R², R³, R⁶, m and Q having the meanings given above, with a succinimide of the formula (III)

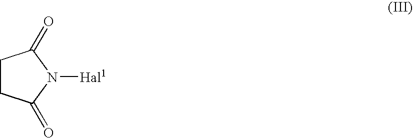

in which

Hal¹ represents chlorine or bromine,

or

A2) Δ¹-pyrrolines of the formula (II-a)

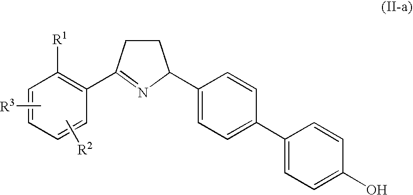

in which

R¹, R², and R³ have the meanings given above, with a sulphonylhalide of the formula (IV)

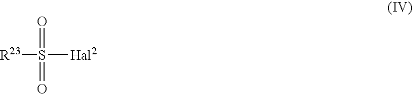

in which

Hal² represents chlorine or bromine,

R²³ represents alkyl or halogenoalkyl, if appropriate in the presence of an acid binder and if appropriate in the presence of a diluent.

B) Δ¹-Pyrrolines of the formula (I-b)

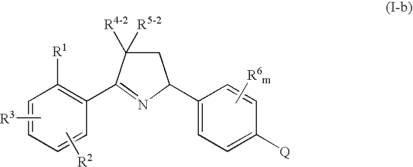

in which

R¹, R², R³, R⁶, m and Q have the meanings given above,

R⁴⁻² represents alkyl or halogenoalkyl,

R⁵⁻² represents hydrogen, alkyl or halogenoalkyl, are obtained by treating aminoketones of the formula (V)

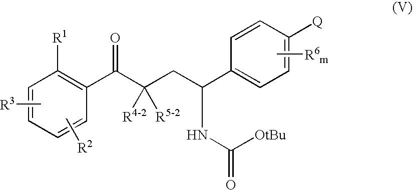

in which

R¹, R², R³, R⁴⁻², R⁵⁻², R⁶, m and Q have the meanings given above, with a Lewis acid or a protonic acid, if appropriate in the presence of a diluent.

C) Δ¹-Pyrrolines of the formula (I-c)

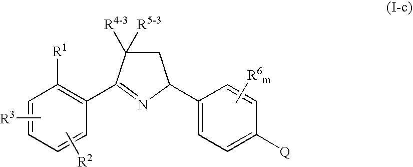

in which

R¹, R², R³, R⁶, m and Q have the meanings given above,

R⁴⁻³ represents —SO₃R⁸, where R⁸ has the meanings given above,

R⁵⁻³ represents hydrogen, are obtained by reacting cyclopropanes of the formula (VI)

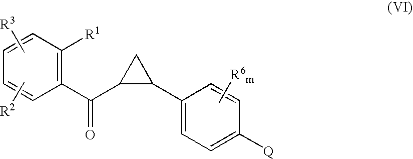

in which

R¹, R², R³, R⁶, m and Q have the meanings given above, with sulphuric acid and acetonitrile and, if appropriate, in the presence of an alcohol of the formula (VII)

R⁸—OH (VII)

in which

R⁸ has the meanings given above.

D) Δ¹-Pyrrolines of the formula (I-d)

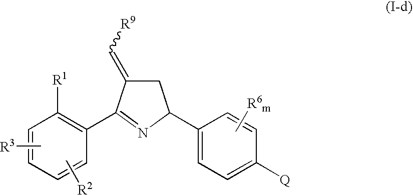

in which

R¹, R², R³, R⁶, m, Q and R⁹ have the meanings given above, are obtained by reacting Δ¹-pyrrolines of the formula (II)

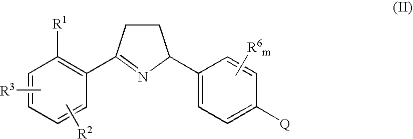

in which

R¹, R², R³, R⁶, m and Q have the meanings given above, with an aldehyde of the formula (VIII)

R⁹—CHO (VIII)

in which

R⁹ has the meanings given above, if appropriate in the presence of a base and if appropriate in the presence of a diluent.

Finally, it has been found that the compounds of the formula (I) according to the invention have very good insecticidal properties and can be used both in crop protection and in the protection of materials for controlling undesirable pests, such as insects.

The formula (I) provides a general definition of the Δ¹-pyrrolines according to the invention.

R¹ preferably represents halogen, alkyl, halogenoalkyl, alkoxy, halogenoalkoxy or —S(O)_{w}R⁷.

R² and R³ independently of one another preferably represent hydrogen, halogen, alkyl, halogenoalkyl, alkoxy, halogenoalkoxy or alkoxyalkyl.

R⁴ preferably represents halogen, alkyl, halogenoalkyl or represents —SO₃R⁸.

R⁵ preferably represents hydrogen, halogen, alkyl, halogenoalkyl or represents —SO₃R⁸.

H⁴ and R⁵ furthermore together preferably represent the grouping

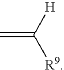

R⁶ preferably represents halogen, alkyl, alkoxy, alkylthio, halogenoalkyl, halogenoalkoxy or halogenoalkylthio.

m preferably represents 0, 1, 2, 3 or 4.

Q preferably represents the grouping —X—Y-Z-E, with the proviso that Y does not represent a direct bond if X does not represent a direct bond.

R⁷ preferably represents alkyl or halogenoalkyl.

R⁸ preferably represents hydrogen, alkyl or halogenoalkyl.

R⁹ preferably represents hydrogen, alkyl, halogenoalkyl or represents aryl or 5- to 10-membered heteroaryl having one or more heteroatoms from the group consisting of nitrogen, oxygen and sulphur, each of which radicals is optionally mono- to tetrasubstituted by identical or different substituents from the list W¹.

X preferably represents a direct bond, oxygen, —S(O)_{w}—, —NR¹⁰—, carbonyl, carbonyloxy, oxycarbonyl, oxysulphonyl (OSO₂), alkylene, halogenoalkylene, alkenylene, halogenoalkenylene, alkinylene, alkylenoxy, oxyalkylene, oxyalkylenoxy, —S(O)_{w}-alkylene, cyclopropylene or oxiranylene.

Y preferably represents a direct bond or represents phenylene, naphthylene, tetrahydronaphthylene or 5- to 10-membered saturated or unsaturated heterocyclylene having one or more heteroatoms from the group consisting of nitrogen, oxygen and sulphur, each of which radicals is optionally mono- to tetrasubstituted by identical or different radicals from the list W¹.

Z preferably represents a direct bond or —(CH₂)ₙ—.

E preferably represents hydrogen, halogen, hydroxyl, cyano, formyl, nitro, trialkylsilyl, pentafluorothio, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —C(R¹⁸)═N—OR¹⁹, —SO₂NR²⁰R²¹; represents alkyl, alkenyl, alkinyl, alkoxy, alkenyloxy, each of which is optionally mono- or polysubstituted by identical or different substituents from the group consisting of halogen, cyano, alkoxy and —NR¹²R¹³; or represents cycloalkyl, cycloalkylalkyl, cycloalkyloxy, aryl, arylalkyl, aryloxy, aryloxyalkyl, saturated or unsaturated 5- to 10-membered heterocyclyl or heterocyclylalkyl having one or more heteroatoms from the group consisting of nitrogen, oxygen and sulphur, each of which radicals is mono- or polysubstituted by identical or different substituents from the group consisting of halogen, cyano, nitro, alkyl, halogenoalkyl, alkenyl, halogenoalkenyl, alkoxy, halogenoalkoxy, alkylthio and halogenoalkylthio.

W¹ preferably represents halogen, cyano, formyl, nitro, trialkylsilyl, alkyl, halogenoalkyl, alkoxy, halogenoalkoxy, alkenyl, halogenoalkenyl, alkenyloxy, halogenoalkenyloxy, alkylcarbonyl, alkoxycarbonyl, —S(O)_{w}R¹¹, —C(R¹⁸)═N—OR¹⁹, —SO₂NR²⁰R²¹, —(CH₂)ₚNR²⁰R²¹, —(CH₂)ₚN(R²⁰)COR²¹, —(CH₂)ₚN(R²⁰)SO₂R²¹, —OSO₂R²⁰, —OSO₂NR²⁰R²¹.

R¹⁰ preferably represents alkyl, halogenoalkyl, or represents cycloalkyl, cycloalkylalkyl, aryl or arylalkyl, each of which is optionally mono- or polysubstituted by identical or different substituents from the group consisting of halogen, alkyl, halogenoalkyl, alkoxy, halogenoalkoxy, alkylthio and halogenoalkylthio.

R¹¹ preferably represents alkyl which is optionally mono- or polysubstituted by identical or different substituents from the group consisting of halogen and —NR¹²R¹³, represents cycloalkyl, aryl or arylalkyl, each of which is optionally mono- or polysubstituted by identical or different substituents from the group consisting of halogen, cyano, alkyl, halogenoalkyl, alkoxy, halogenoalkoxy, alkylthio and halogenoalkylthio.

R¹² and R¹³ independently of one another preferably represent hydrogen or —SO₂R¹¹, represent alkyl or alkenyl, each of which is optionally mono- or polysubstituted by identical or different substituents from the group consisting of halogen, alkylcarbonyl, alkylcarbonyloxy, alkylamino, dialkylamino, alkoxy, halogenoalkoxy, alkylthio and halogenoalkylthio; represents cycloalkyl, cycloalkylalkyl, aryl, arylalkyl, saturated or unsaturated 5- to 10-membered heterocyclyl or heterocyclylalkyl having one or more heteroatoms from the group consisting of nitrogen, oxygen and sulphur, each of which radicals is optionally mono- or polysubstituted by identical or different substituents from the group consisting of halogen, cyano, alkyl, halogenoalkyl, alkoxy, halogenoalkoxy, alkylthio and halogenoalkylthio.

R¹² and R¹³ furthermore together preferably represent alkenylene which is optionally mono- or polysubstituted by identical or different substituents from the group consisting of halogen, cyano, alkoxy, halogenoalkoxy, alkylthio and halogenoalkylthio or represent alkylene, where the alkylene chain may be interrupted by —O—, —S— or —NR²²—, and where the radical is optionally mono- or polysubstituted by identical or different substituents from the group consisting of halogen, cyano, alkyl, halogenoalkyl, alkoxy, halogenoalkoxy, alkylthio and halogenoalkylthio.

R¹⁴ and R¹⁵ independently of one another preferably represent hydrogen, —SO₂R¹¹, represent alkyl or alkenyl, each of which is optionally mono- or polysubstituted by identical or different substituents from the group consisting of halogen, alkylamino, dialkylamino, alkoxy and alkylthio; represents cycloalkyl, cycloalkylalkyl, aryl, arylalkyl, saturated or unsaturated 5- to 10membered heterocyclyl or heterocyclylalkyl having one or more heteroatoms from the group consisting of nitrogen, oxygen and sulphur, each of which radicals is optionally mono- or polysubstituted by identical or different substituents from the group consisting of halogen, cyano, alkyl, halogenoalkyl, alkoxy, halogenoalkoxy, alkylthio and halogenoalkylthio.

R¹⁴ and R¹⁵ furthermore together preferably represent alkylene, where the alkylene chain may be interrupted by —O—, —S— or —NR²²—, and where the radical is optionally mono- or polysubstituted by identical or different substituents from the group consisting of halogen, cyano, alkyl, halogenoalkyl, alkoxy, halogenoalkoxy, alkylthio and halogenoalkylthio.

R¹⁶ and R¹⁷ independently of one another preferably represent hydrogen, represent alkyl which is optionally mono- or polysubstituted by identical or different substituents from the group consisting of halogen, cyano, alkoxy and alkylthio, represent cycloalkyl, cycloalkylalkyl, aryl or arylalkyl, each of which is optionally mono- or polysubstituted by identical or different substituents from the group consisting of halogen, cyano, alkyl, halogenoalkyl, alkoxy, halogenoalkoxy, alkylthio and halogenoalkylthio.

R¹⁶ and R¹⁷ furthermore together preferably represent alkylene or alkenylene, each of which is optionally mono- or polysubstituted by identical or different substituents from the group consisting of halogen, alkyl, halogenoalkyl, alkoxy, halogenoalkoxy, alkylthio and halogenoalkylthio.

R¹⁸ and R¹⁹ independently of one another preferably represent hydrogen, alkyl, alkenyl, halogenoalkyl or halogenoalkenyl.

R²⁰ and R²¹ independently of one another preferably represent hydrogen, alkyl, halogenoalkyl or represent cycloalkyl which is optionally mono- or polysubstituted by identical or different substituents from the group consisting of halogen and alkyl

R²⁰ and R²¹ furthermore together preferably represent alkylene, alkoxyalkylene or alkylthioalkylene, each of which is optionally mono- or polysubstituted by identical or different substituents from the group consisting of halogen and alkyl.

R²² preferably represents hydrogen, —SO₂R¹¹, —COR¹¹ or —CO₂R¹¹; represents alkyl or alkenyl, each of which is optionally mono- or polysubstituted by identical or different substituents from the group consisting of halogen, cyano, alkylamino, dialkylamino, alkoxy, halogenoalkoxy, alkylthio and halogenoalkylthio; represents cycloalkyl, cycloalkylalkyl, aryl, arylalkyl, saturated or unsaturated 5- to 10-membered heterocyclyl or heterocyclylalkyl having one or more heteroatoms from the group consisting of nitrogen, oxygen and sulphur, each of which radicals is optionally mono- or polysubstituted by identical or different substituents from the group consisting of halogen, cyano, alkyl, halogenoalkyl, alkoxy, halogenoalkoxy, alkylthio and halogenoalkylthio.

w preferably represents 0, 1 or 2.

n preferably represents 1, 2, 3 or 4.

p preferably represents 0, 1 or 2.

R¹ particularly preferably represents halogen, C₁-C₆-alkyl, C₁-C₆-halogenoalkyl, C₁-C₆-alkoxy, C₁-C₆-halogenoalkoxy or —S(O)_{w}R⁷.

R² and R³ independently of one another particularly preferably represent hydrogen, halogen, C₁-C₆-alkyl, C₁-C₆-halogenoalkyl, C₁-C₆-alkoxy, C₁-C₆-halogenoalkoxy or C₁-C₆-alkoxy-C₁-C₆-alkyl.

R⁴ particularly preferably represents halogen, C₁-C₁₀-alkyl, C₁-C₁₀halogenoalkyl or represents —SO₃R⁸.

R⁴ and R⁵ furthermore together particularly preferably represent the grouping

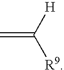

R⁵ particularly preferably represents hydrogen, halogen, C₁-C₁₀-alkyl, C₁-C₁₀-halogenoalkyl or represents —SO₃R⁸.

R⁶ particularly preferably represents fluorine, chlorine, bromine, C₁-C₆-alkyl, C₁-C₆-alkoxy, C₁-C₆-alkylthio, C₁-C₆-halogenoalkyl, C₁-C₆-halogenoalkoxy or C₁-C₆-halogenoalkylthio.

m particularly preferably represents 0, 1, 2 or 3.

Q particularly preferably represents the grouping —X—Y-Z-E, with the proviso that Y does not represent a direct bond if X does not represent a direct bond.

R⁷ particularly preferably represents C₁-C₆-alkyl or C₁-C₆-halogenoalkyl.

R⁸ particularly preferably represents hydrogen, C₁-C₆-alkyl or C₁-C₆halogenoalkyl.

R⁹ particularly preferably represents hydrogen, C₁-C₆-alkyl, C₁-C₆-halogenoalkyl, or represents aryl or 5- or 6-membered heteroaryl having I to 3 heteroatoms, which -contains 0 to 3 nitrogen atoms, 0 to 2 non-adjacent oxygen atoms and 0 to 2 non-adjacent sulphur atoms (in particular furyl, thienyl, pyrrolyl, oxazolyl, thiazolyl or pyridinyl), each of which radicals is optionally mono- to tetrasubstituted by identical or different radicals from the list W¹.

X particularly preferably represents a direct bond, oxygen, —S(O)_{w}—, —NR¹⁰—, carbonyl, carbonyloxy, oxycarbonyl, oxysulphonyl (OSO₂), C₁-C₆-alkylene, C₁-C₆-halogenoalkylene, C₂-C₆-alkenylene, C₂-C₆-halogenoalkenylene, C₂-C₆-alkinylene, C₁-C₆-alkylenoxy, C₁-C₆-oxyalkylene, oxy-C₁-C₆-alkylenoxy or —S(O)_{w}—C₁-C₆-alkylene.

Y particularly preferably represents a direct bond or represents 1,4-phenylene, 1,3-phenylene, 1,2-phenylene, 2,6-naphthylene, 2,7-naphthylene, 1,4-naphthylene, 2,6-(1,2,3,4-tetrahydro)naphthylene, 2,7-(1,2,3,4-tetrahydro)naphthylene, 1,4-(1,2,3,4-tetrahydro)naphthylene, 5,8-(1,2,3,4-tetrahydro)naphthylene, each of which is optionally mono- to tetrasubstituted by identical or different radicals from the list W¹; or represents 5- or 6-membered saturated or unsaturated heterocyclylene having 1 to 3 heteroatoms, which contains 0 to 3 nitrogen atoms, 0 to 2 non-adjacent oxygen atoms and/or 0 to 2 non-adjacent sulphur atoms (in particular furylene, thienylene, pyrrolylene, oxazolylene, thiazolylene, pyridinylene, pyrimidinylene or pyridazinylene or pyrazinylene), each of which radicals is optionally mono- to tetrasubstituted by identical or different radicals from the list W¹.

Z particularly preferably represents a direct bond or —(CH₂)ₙ—.

E particularly preferably represents hydrogen, fluorine, chlorine, bromine, hydroxyl, cyano, formyl, nitro, tri-(C₁-C₆-alkyl)silyl, pentafluorothio, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —C(R¹⁸)═N—OR¹⁹, —SO₂NR²⁰R²¹; represents C₁-C₂₀-alkyl, C₂-C₂₀-alkenyl, C₂-C₁₀-alkinyl, C₁-C₂₀-alkoxy, C₂-C₂₀-alkenyloxy, each of which is optionally mono- or polysubstituted by identical or different substituents from the group consisting of halogen, cyano, C₁-C₁₀-alkoxy and —NR¹²R¹³; or represents C₃-C₁₂-cycloalkyl, C₃-C₇-cycloalkyl-C₁-C₄-alkyl, C₃-C₁₂-cycloalkyloxy, aryl, aryl-C₁-C₄-alkyl, aryloxy, aryloxy-C₁-C₄-alkyl, saturated or unsaturated 5- to 10-membered heterocyclyl or heterocyclyl-C₁-C₄-alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrabydrofuryl, thienyl, thenyl, benzothienyl, thiolanyl, pyrrolyl, indolyl, pyrrolinyl, pyrrolidino, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, piperidino, morpholinyl, thiomorpholinyl, morpholino, thiomorpholino, triazinyl, triazolyl, quinolinyl or isoquinolinyl), each of which radicals is optionally mono- to tetrasubstituted by identical or different radicals from the group consisting of halogen, cyano, nitro, C₁-C₆-alkyl, C₁-C₆-halogenoalkyl, C₂-C₆-alkenyl, C₂-C₆-halogenoalkenyl, C₁-C₆-alkoxy, C₁-C₆-halogenoalkoxy, C₁-C₆-alkylthio and C₁-C₆-halogenoalkylthio.

W¹ particularly preferably represents fluorine, chlorine, bromine, cyano, formyl, nitro, tri-(C₁-C₆-alkyl)silyl, C₁-C₆-alkyl, C₁-C₆-halogenoalkyl, C₁-C₆-alkoxy, C₁-C₆-halogenoalkoxy, C₂-C₆-alkenyl, C₂-C₆-halogenoalkenyl, C₂-C₆-alkenyloxy, C₂-C₆-halogenoalkenyloxy, C₁-C₆-alkylcarbonyl, C₁-C₆-alkoxycarbonyl, —S(O)_{w}R¹¹, —C(R¹⁸)═N—OR¹⁹, —SO₂NR²⁰R²¹, —(CH₂)ₚNR²⁰R²¹, —(CH₂)ₚN(R²⁰)COR²¹, —(CH₂)ₚN(R²⁰)SO₂R²¹, —OSO₂R²⁰, —OSO₂NR²⁰R²¹.

R¹⁰ particularly preferably represents C₁-C₆-alkyl, C₁-C₆-halogenoalkyl, or represents C₃-C₇-cycloalkyl, C₃-C₆-cycloalkyl-C₁-C₄-alkyl, aryl or aryl-C₁-C₄-alkyl, each of which is mono- to tetrasubstituted by identical or different substituents from the group consisting of halogen, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹¹ particularly preferably represents C₁-C₂₀-alkyl, each of which is optionally mono- or polysubstituted by identical or different substituents from the group consisting of halogen and —NR¹²R¹³, represents C₃-C₆-cycloalkyl, aryl or aryl-C₁-C₄-alkyl, each of which is optionally mono- to octasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹² and R¹³ independently of one another particularly preferably represent hydrogen, —SO₂R¹¹, represent C₁-C₂₀-alkyl or C₂-C₂₀-alkenyl, each of which optionally is mono- or polysubstituted by identical or different substituents from the group consisting of halogen, C₁-C₆-alkylcarbonyl, C₁-C₆-alkylcarbonyloxy, C₁-C₆-alkylamino, di-(C₁-C₆-alkyl)amino, C₁-C₆-alkoxy, C₁-C₆-halogenoalkoxy, C₁-C₆-alkylthio and C₁-C₆-halogenoalkylthio; represent C₃-C₁₂-cycloalkyl, C₃-C₇-cycloalkyl-C₁-C₄-alkyl, aryl, aryl-C₁-C₄-alkyl, saturated or unsaturated 5- to 10-membered heterocyclyl or heterocyclyl-C₁-C₄-alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl), each of which radicals is optionally mono- to tetrasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹² and R¹³ furthermore together particularly preferably represent C₂-C₁₂-alkenylene which is optionally mono- or polysubstituted by identical or different substituents from the group consisting of halogen, cyano, C₁-C₆-alkoxy, C₁-C₆-halogenoalkoxy, C₁-C₆-alkylthio and C₁-C₆-halogenoalkylthio or represent C₃-C₁₂-alkylene, where the alkylene chain may be interrupted by —O—, —S— or —NR²²—, and where the radical is optionally mono- or polysubstituted in the alkylene moiety by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹⁴ and R¹⁵ independently of one another particularly preferably represent hydrogen, —SO₂R¹¹, represent C₁-C₆-alkyl or C₂-C₆-alkenyl, each of which is optionally mono- to tridecasubstituted by identical or different substituents from the group consisting of halogen, C₁-C₆-alkylamino, di-(C₁-C₆-alkyl)amino, C₁-C₆-alkoxy, C₁-C₆-halogenoalkoxy, C₁-C₆-alkylthio and C₁-C₆-halogenoalkylthio; represent C₃-C₇-cycloalkyl, C₃-C₇-cycloalkyl-C₁-C₄-alkyl, aryl, aryl-C₁-C₄-alkyl, saturated or unsaturated 5- to 10-membered heterocyclyl or heterocyclyl-C₁-C₄-alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl), each of which is optionally mono- to tetrasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹⁴ and R¹⁵ furthermore together particularly preferably represent C₃-C₆-alkylene which is optionally mono- to tetrasubstituted in the alkylene moiety by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio, represent —(CH₂)₂—O—(CH₂)₂—, —(CH₂)₂—S—(CH₂)₂— or —(CH₂)₂—N(R²²)—(CH₂)₂—.

R¹⁶ and R¹⁷ independently of one another particularly preferably represent hydrogen, represent C₁-C₆-alkyl which is optionally mono- to tridecasubstituted by identical or different substituents from the group consisting of halogen, cyano, C₁-C₆-alkoxy and C₁-C₆-alkylthio, represent C₃-C₇-cycloalkyl, C₃-C₇-cycloalkyl-C₁-C₄-alkyl, aryl or aryl-C₁-C₄-alkyl, each of which is optionally mono- to octasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹⁶ and R¹⁷ furthermore together particularly preferably represent C₃-C₁₀-alkylene or C₃-C₁₀-alkenylene, each of which is optionally mono- to octasubstituted by identical or different substituents from the group consisting of halogen, C₁C₆-alkyl, C₁-C₆-halogenoalkyl, C₁-C₆-alkoxy, C₁-C₆-halogenoalkoxy, C₁-C₆-alkylthio and C₁-C₆-halogenoalkylthio.

R¹⁸ and R¹⁹ independently of one another particularly preferably represent hydrogen, C₁-C₆-alkyl, C₂-C₆-alkenyl, C₁-C₆-halogenoalkyl or C₂-C₆-halogenoalkenyl.

R²⁰ and R²¹ independently of one another particularly preferably represent hydrogen, C₁-C₆-alkyl, C₁-C₆-halogenoalkyl or represent C₃-C₇-cycloalkyl which is optionally mono- to octasubstituted by identical or different substituents from the group consisting of fluorine, chlorine, bromine and C₁-C₆-alkyl.

R²⁰ and R²¹ furthermore together particularly preferably represent C₃-C₆-alkylene, C₁-C₃-alkoxy-C₁-C₃-alkylene or C₁-C₃-alkylthio-C₁-C₃-alkylene, each of which is optionally mono- to nonasubstituted by identical or different substituents from the group consisting of fluorine, chlorine, bromine and C₁-C₆-alkyl.

R²² particularly preferably represents hydrogen, —SO₂R¹¹, —COR¹¹ or —CO₂R¹¹; represents C₁-C₂₀-alkyl or C₂-C₂₀-alkenyl, each of which is optionally mono- or polysubstituted by identical or different substituents from the group consisting of halogen, cyano, C₁-C₆-alkylamino, di-(C₁-C₆-alkyl)amino, C₁-C₆-alkoxy, C₁-C₆-halogenoalkoxy, C₁-C₆-alkylthio and C₁-C₆-halogenoalkylthio; represents C₃-C₁₂-cycloalkyl, C₃-C₇-cycloalkyl-C₁-C₄-alkyl, aryl, aryl-C₁-C₄-alkyl, saturated or unsaturated 5- to 10-membered heterocyclyl or heterocyclyl-C₁-C₄-alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl), each of which radicals is optionally mono- to tetrasubstituted by identical or different radicals from the group consisting of halogen, cyano, C₁-C₆-alkyl, C₁-C₆-halogenoalkyl, C₁-C₆alkoxy, C₁-C₆-halogenoalkoxy, C₁-C₆-alkylthio and C₁-C₆-halogenoalkylthio.

w particularly preferably represents 0, 1 or 2.

n particularly preferably represents 1, 2 or 3.

p particularly preferably represents 0, 1 or 2.

R¹ very particularly preferably represents fluorine, chlorine, bromine, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms or —S(O)_{w}R⁷.

R² and R³ independently of one another very particularly preferably represent hydrogen, fluorine, chlorine, bromine, C₁-C₄-alkyl, C₁-C₄-halogenoalkyl having 1 to 9 fluorine, chlorine and/or bromine atoms, C₁-C₄-alkoxy or C₁-C₄-halogenoalkoxy having 1 to 9 fluorine, chlorine and/or bromine atoms.

R⁴ very particularly preferably represents fluorine, chlorine, bromine, C₁-C₆-alkyl, C₁-C₆-halogenoalkyl having 1 to 13 fluorine, chlorine and/or bromine atoms, or represents —SO₃R⁸.

R⁵ very particularly preferably represents hydrogen, fluorine, chlorine, bromine, C₁-C₆-alkyl, C₁-C₆-halogenoalkyl having 1 to 13 fluorine, chlorine and/or bromine atoms, or represents —SO₃R⁸.

R⁴ and R⁵ furthermore together very particularly preferably represent the grouping

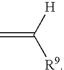

R⁶ very particularly preferably represents fluorine, chlorine, C₁-C₄-alkyl, C₁-C₄-alkoxy, C₁-C₄-alkylthio; C₁-C₄-halogenoalkyl, C₁-C₄-halogenoalkoxy, C₁-C₄-halogenoalkylthio having in each case 1 to 9 fluorine, chlorine and/or bromine atoms.

m very particularly preferably represents 0, 1 or 2.

Q very particularly preferably represents the grouping —X—Y-Z-E, with the proviso that Y does not represent a direct bond if X does not represent a direct bond.

R⁷ very particularly preferably represents C₁-C₄-alkyl or represents in each case fluorine- or chlorine-substituted methyl or ethyl.

R⁸ very particularly preferably represents hydrogen, C₁-C₄-alkyl or C₁-C₄-halogenoalkyl having 1 to 9 fluorine, chlorine and/or bromine atoms.

R⁹ very particularly preferably represents hydrogen, C₁-C₄-alkyl, C₁-C₄-halogenoalkyl having 1 to 9 fluorine, chlorine and/or bromine atoms, or represents phenyl, furyl, thienyl, pyrrolyl, oxazolyl, thiazolyl or pyridinyl, each of which is optionally mono- to trisubstituted by radicals from the list W¹.

X very particularly preferably represents a direct bond, oxygen, —S(O)_{w}—, —NR¹⁰—, carbonyl, carbonyloxy, oxycarbonyl, oxysulphonyl (OSO₂), C₁-C₄-alkylene, C₁-C₄-halogenoalkylene having 1 to 8 fluorine, chlorine and/or bromine atoms, C₂-C₄-alkenylene, C₂-C₄-halogenoalkenylene having 1 to 6 fluorine, chlorine and/or bromine atoms, C₂-C₄-alkinylene, C₁-C₄-alkylenoxy, oxy-C₁-C₄-alkylene, oxy-C₁-C₄-alkylenoxy or —S(O)_{w}—C₁-C₄-alkylene.

Y very particularly preferably represents a direct bond or represents 1,4-phenylene, 1,3-phenylene, 1,2-phenylene, 2,6-naphthylene, 2,7-naphthylene, 1,4-naphthylene, 2,6-(1,2,3,4-tetrahydro)naphthylene, 2,7-(1,2,3,4-tetrahydro)naphthylene, 1,4-(1,2,3,4-tetrahydro)naphthylene, 5,8-(1,2,3,4-tetrahydro)naphthylene, 2,4-furylene, 2,4-thienylene, 2,4-pyrrolylene, 2,5-oxazolylene, 2,5-thiazolylene, 2,5-pyridinylene, 2,6-pyridinylene, 2,5-pyrimidinylene or 3,6-pyridazinylene or 2,5-pyrazinylene, each of which is optionally mono- to trisubstituted by identical or different radicals from the list W¹.

Z very particularly preferably represents a direct bond or —(CH₂)ₙ—.

E very particularly preferably represents hydrogen, fluorine, chlorine, bromine, hydroxyl, cyano, formyl, nitro, trimethylsilyl, dimethyl-tert-butylsilyl, —S(O)_{w}R¹¹, —OSO₂R¹¹, —NR¹²R¹³, —COR¹¹, —CO₂R¹¹, —OC(O)R¹¹, —-CONR¹⁴R¹⁵, —N(R¹⁶)COR¹⁷, —SO₂NR²⁰R²¹; represents C₁-C₁₆-alkyl, C₂-C₁₆-alkenyl, C₂-C₆alkinyl, C₁-C₁₆-alkoxy, C₂-C₁₆-alkenyloxy, each of which is optionally mono- or polysubstituted by identical or different radicals from the group consisting of fluorine, chlorine, bromine, cyano, C₁-C₆-alkoxy and —NR¹²R¹³; or represents C₃-C₁₀-cycloalkyl, C₃-C₆-cycloalkyl-C₁-C₄-alkyl, C₃-C₁₀-cycloalkyloxy, phenyl, phenoxy, benzyl, phenylethyl, benzyloxy, tetrazolyl, furyl, furfuryl, benzofuryl, tetrahydrofuryl, thienyl, thenyl, benzothienyl, thiolanyl, pyrrolyl, indolyl, pyrrolinyl, pyrrolidino, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, piperidino, morpholinyl, thiomorpholinyl, morpholino, thiomorpholino, triazinyl, triazolyl, quinolinyl or isoquinolinyl, each of which is optionally mono- to trisubstituted by identical or different radicals from the group consisting of fluorine, chlorine, bromine, cyano, C₁-C₄-alkyl, C₁-C₄-halogenoalkyl having 1 to 9 fluorine, chlorine and/or bromine atoms, C₂-C₆-alkenyl, C₂-C₆-halogenoalkenyl having 1 to 8 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

W¹ very particularly preferably represents fluorine, chlorine, bromine, cyano, formyl, trimethylsilyl, dimethyl-tert-butylsilyl, C₁-C₄-alkyl, C₁-C₄-alkoxy, C₂-C₄-alkenyl, C₂-C₄-alkenyloxy; represents C₁-C₄-halogenoalkyl, C₁-C₄-halogenoalkoxy having in each case 1 to 9 fluorine, chlorine and/or bromine atoms, C₂-C₄-halogenoalkenyl, C₂-C₄-halogenoalkenyloxy having in each case 1 to 8 fluorine, chlorine and/or bromine atoms; represents C₁-C₄-alkylcarbonyl, C₁-C₄-alkoxycarbonyl, —S(O)_{w}R¹¹, —SO₂NR²⁰R²¹, —(CH₂)ₚNR²⁰R²¹, —(CH₂)ₚN(R²⁰)COR²¹, —(CH₂)ₚN(R²⁰)SO₂R²¹, —OSO₂R²⁰, —OSO₂NR²⁰R²¹.

R¹⁰ very particularly preferably represents methyl, ethyl, n-propyl, isopropyl, n-butyl, isobutyl, sec-butyl, tert-butyl, trifluoromethyl, trifluoroethyl, or represents cyclopropyl, cyclopropylmethyl, cyclopentyl, cyclopentylmethyl, cyclohexyl, cyclohexylmethyl, phenyl, benzyl or phenylethyl, each of which is optionally mono- to tetrasubstituted by identical or different substituents from the group consisting of fluorine, chlorine, bromine, C₁-C₄-alkyl, C₁-C₄-alkoxy, C₁-C₄-alkylthio and C₁-C₄-halogenoalkyl, C₁-C₄-halogenoalkoxy and C₁-C₄-halogenoalkylthio having in each case 1 to 9 fluorine, chlorine and/or bromine atoms.

R¹¹ very particularly preferably represents C₁-C₁₀-alkyl which is optionally mono- or polysubstituted by identical or different substituents from the group consisting of fluorine, chlorine, bromine and —NR¹²R¹³, represents cyclopropyl, cyclopentyl, cyclohexyl, phenyl or benzyl, each of which is optionally mono- to tetrasubstituted by identical or different substituents from the group consisting of fluorine, chlorine, bromine, cyano, C₁-C₄-alkyl, C₁-C₄-alkoxy, C₁-C₄-alkylthio, C₁-C₄-halogenoalkyl, C₁-C₄-halogenoalkoxy, and C₁-C₄-halogenoalkylthio having in each case 1 to 9 fluorine, chlorine and/or bromine atoms.

R¹² and R¹³ independently of one another very particularly preferably represent hydrogen, —SO₂R¹¹, represent C₁-C₁₆-alkyl or C₂-C₁₆-alkenyl, each of which is optionally mono- or polysubstituted by identical or different substituents from the group consisting of fluorine, chlorine, bromine, C₁-C₄-alkylcarbonyl, C₁-C₄-alkylcarbonyloxy, C₁-C₄-alkylamino, di-(C₁-C₄-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₁₀-cycloalkyl, C₃-C₆-cycloalkyl-C₁-C₄-alkyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R¹² and R¹³ furthermore together very particularly preferably represent C₂-C₁₀-alkenylene, which is optionally mono- or polysubstituted by identical or different substituents from the group consisting of fluorine, chlorine, bromine, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms or represents C₃-C₁₀-alkylene, where the alkylene chain may be interrupted by —O—, —S— or —NR²²—, and where the radicals are optionally mono- or polysubstituted in the alkylene moiety by identical or different substituents from the group consisting of fluorine, chlorine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R¹⁴ and R¹⁵ independently of one another very particularly preferably represent hydrogen, —SO₂R¹¹, represent C₁-C₆-alkyl or C₂-C₆-alkenyl, each of which is optionally mono- to nonasubstituted by identical or different substituents from the group consisting of fluorine, chlorine, bromine, C₁-C₄-alkylamino, di-(C₁-C₄-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₆-cycloalkyl, C₃-C₆-cycloalkyl-C₁-C₄-alkyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, C₁-C₄alkyl, cyano, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R¹⁴ and R¹⁵ furthermore together very particularly preferably represent C₄-C₅-alkylene, —(CH₂)₂—O—(CH₂)₂—, —(CH₂)₂—S—(CH₂)₂—, —(CH₂)₂—N(R²²)—(CH₂)₂— is optionally mono- to tetrasubstituted in the alkylene moiety by identical or different substituents from the group consisting of fluorine, chlorine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R¹⁶ and R¹⁷ independently of one another very particularly preferably represent hydrogen, represent C₁-C₆-alkyl which is optionally mono- to nonasubstituted by identical or different substituents from the group consisting of fluorine, chlorine, bromine, C₁-C₄-alkoxy and C₁-C₄-alkylthio, represent C₃-C₆-cycloalkyl, C₃-C₆-cycloalkyl-C₁-C₄-alkyl, phenyl, benzyl or phenylethyl, each of which is optionally mono- to tetrasubstituted by identical or different substituents from the group consisting of fluorine, chlorine, bromine, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R¹⁶ and R¹⁷ furthermore together very particularly preferably represent C₃-C₈-alkylene or C₃-C₈-alkenylene, each of which is optionally mono- to tetrasubstituted by identical or different substituents from the group consisting of fluorine, chlorine, bromine, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R²⁰ and R²¹ independently of one another very particularly preferably represent hydrogen, C₁-C₄-alkyl, C₁-C₄-halogenoalkyl having 1 to 9 fluorine, chlorine and/or bromine atoms or represent C₃-C₆-cycloalkyl which is optionally mono- to tetrasubstituted by identical or different substituents from the group consisting of fluorine, chlorine, bromine and C₁-C₄-alkyl.

R²⁰ and R²¹ furthermore together very particularly preferably represent —(CH₂)₃—, —(CH₂)₄—, —(CH₂)₅—, —(CH₂)₂—O—(CH₂)₂— or —(CH₂)₂—S—(CH₂)₂—.

R²² very particularly preferably represents hydrogen, —SO₂R¹¹, represents —COR¹¹ or —CO₂R¹¹; represents C₁-C₁₆-alkyl or C₂-C₁₆-alkenyl, each of which is optionally mono- or polysubstituted by identical or different substituents from the group consisting of fluorine, chlorine, bromine, cyano, methylamino, ethylamino, di-(C₁-C₆-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₁₀-cycloalkyl, C₃-C₆-cycloalkyl-C₁-C₄-alkyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted with identical or different substituents from the group consisting of fluorine, chlorine, bromine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

w very particularly preferably represents 0, 1 or 2.

n very particularly preferably represents 1 or 2.

p very particularly preferably represents 0 or 1.

R¹ especially preferably represents fluorine, chlorine, bromine, methyl, trifluoromethyl, methoxy, trifluoromethoxy, methylthio or trifluoromethylthio.

R² and R³ independently of one another especially preferably represent hydrogen, fluorine, chlorine, bromine, methyl, trifluoromethyl, methoxy or trifluoromethoxy.

R⁴ especially preferably represents chlorine, bromine, methyl, ethyl, n-propyl, isopropyl, n-butyl, isobutyl, sec-butyl, tert-butyl, trifluoromethyl, trifluoroethyl or represents —SO₃R⁸.

R⁵ especially preferably represents hydrogen, chlorine, bromine, methyl, ethyl, n-propyl, isopropyl, n-butyl, isobutyl, sec-butyl, tert-butyl, trifluoromethyl, trifluoroethyl or represents —SO₃R⁸.

R⁴ and R⁵ furthermore together especially preferably represent the grouping

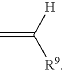

R⁶ especially preferably represents fluorine, chlorine, methyl, ethyl, n-propyl, isopropyl, n-butyl, isobutyl, sec-butyl, tert-butyl, trifluoromethyl, trifluoroethyl, methoxy, ethoxy, n-propoxy, isopropoxy, n-butoxy, isobutoxy, sec-butoxy, tert-butoxy, trifluoromethoxy, trifluoroethoxy, methylthio, ethylthio, n-propylthio, isopropylthio, n-butylthio, isobutylthio, sec-butylthio, tert-butylthio, trifluoromethylthio or trifluoroethylthio.

m especially preferably represents 0 or 1.

Q especially preferably represents the grouping —X—Y-Z-E, with the proviso that Y does not represent a direct bond if X does not represent a direct bond.

R⁸ especially preferably represents hydrogen, methyl, ethyl, n-propyl, isopropyl, trifluoromethyl or trifluoroethyl.

R⁹ especially preferably represents hydrogen, methyl, ethyl, n-propyl, isopropyl, trifluoromethyl, trifluoroethyl, or represents phenyl, furyl, thienyl, pyrrolyl or pyridinyl, each of which is optionally mono- to trisubstituted by identical or different radicals from the list W¹.

X especially preferably represents a direct bond, oxygen, sulphur, —SO₂—, —NR¹⁰—, —CO—, —C(O)—O—, —O—C(O)—, —CH₂—, —(CH₂)₂—, —C═C— (E or Z), —C≡C—, —CH₂O—, —(CH₂)₂O—, —OCH₂—, —O(CH₂)₂—, —O—CH₂—O—, —SCH₂—, —S(CH₂)₂—, —CH₂S— or —(CH₂)₂S—.

Y especially preferably represents a direct bond or represents 1,4-phenylene, 1,3-phenylene, 2,6-naphthylene, 2,7-naphthylene, 2,4-furylene, 2,4-thienylene, 2,5-pyridinylene, 2,5-pyrimidinylene, 3,6-pyridazinylene or 2,5-pyrazinylene, each of which is optionally mono- or disubstituted by identical or different radicals from the list W¹.

Z especially preferably represents a direct bond, methylene or ethylene.

E especially preferably represents hydrogen, fluorine, chlorine, bromine, hydroxyl, cyano, formyl, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —SO₂NR²⁰R²¹; represents C₁-C₁₆-alkyl, C₂-C₁₆-alkenyl, C₁-C₁₆-alkoxy, C₂-C₁₆-alkenyloxy, each of which is optionally mono- or polysubstituted by identical or different radicals from the group consisting of fluorine, chlorine, bromine, cyano, C₁-C₆-alkoxy and —NR¹²R¹³; or represents cyclopropyl, cyclopentyl, cyclohexyl, cyclopropyloxy, cyclopentyloxy, cyclohexyloxy, cyclopropylmethyl, cyclopentylmethyl, cyclohexylmethyl, phenyl, phenoxy, benzyl, phenylethyl, benzyloxy, tetrazolyl, furyl, furfuryl, benzofuryl, tetrahydrofuryl, thienyl, thenyl, benzothienyl, thiolanyl, pyrrolyl, indolyl, pyrrolinyl, pyrrolidino, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, piperidino, morpholinyl, thiomorpholinyl, morpholino, thiomorpholino, triazinyl, triazolyl, quinolinyl or isoquinolinyl, each of which is optionally mono- to trisubstituted by identical or different radicals from the group consisting of fluorine, chlorine, bromine, cyano, methyl, ethyl, n-propyl, isopropyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, vinyl, allyl, 1-propenyl, butenyl, —CF═CHF, —CF═CH₂, —CF═CCl₂, —CH═CF₂, —CF₂CF═CF₂, —CH═CFH, —CH₂CF═CF₂, —CF═CF₂, —CF₂CH═CF₂, methoxy, ethoxy, n-propoxy, isopropoxy, n-butoxy, isobutoxy, sec-butoxy, tert-butoxy, trifluoromethoxy, difluoromethoxy, chlorodifluoromethoxy, trifluoroethoxy, methylthio, ethylthio, n-propylthio, isopropylthio, n-butylthio, isobutylthio, sec-butylthio, tert-butylthio, trifluoromethylthio, difluoromethylthio, chlorodifluoromethylthio, trifluoroethylthio.

W¹ especially preferably represents fluorine, chlorine, bromine, cyano, formyl, methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy, tert-butoxy, vinyl, allyl, trifluoromethyl, trifluoroethyl, trifluoromethoxy, trifluoroethoxy, —OCF₂CF₂H, —CH═CF₂, —CH═CCl₂, —OCF═CF₂, —COMe, —COEt, —CO₂Me, —CO₂Et, —CO₂(t-Bu), —SMe, —SOMe, —SO₂Me, —SCF₃, —SOCF₃, —SO₂CF₃, —SCHF₂, —SOCHF₂, —SO₂CHF₂, —SO₂NMe₂, —NMe₂, —NEt₂—N(n-Pr)₂, —N(Me)COMe, —N(Me)COEt, —N(Me)COPr, —N(Me)CO(t-Bu), 2-pyrrolidon-5-yl, 2-piperidon-6-yl, —N(Me)SO₂Me, —N(Me)SO₂Et, —N(Me)SO₂CF₃, —N(Et)SO₂CF₃, —N(Me)SO₂(CF₂)₃CF₃ or —OSO₂NMe₂.

R¹⁰ especially preferably represents methyl, ethyl, n-propyl, isopropyl, n-butyl, isobutyl, sec-butyl, tert-butyl, trifluoromethyl, trifluoroethyl, cyclopropyl, cyclopropylmethyl, cyclopentyl, cyclopentylmethyl, cyclohexyl, cyclohexylmethyl, phenyl, benzyl or phenylethyl.

R¹¹ especially preferably represents methyl, ethyl, n-propyl, isopropyl, n-butyl, isobutyl, sec-butyl, tert-butyl, pentyl, hexyl, —CF₃, —CHF₂, —CCl₃, —CCl₂F, dimethylaminomethyl, dimethylaminoethyl, diethylaminomethyl, diethylaminoethyl, cyclopropyl, cyclopentyl, cyclohexyl, phenyl or benzyl.

R¹² and R¹³ independently of one another especially preferably represent hydrogen, —SO₂R¹¹, represent C₁-C₁₆-alkyl, C₂-C₁₆-alkenyl, each of which is optionally mono- or polysubstituted by identical or different substituents from the group consisting of fluorine, chlorine, bromine, C₁-C₄-alkylcarbonyl, C₁-C₄-alkylcarbonyloxy, C₁-C₄-alkylamino, di-(C₁-C₄-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₈-cycloalkyl, cyclopropylmethyl, cyclopentylmethyl, cyclohexylmethyl, cyclopropylethyl, cyclopentylethyl, cyclohexylethyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms.

R¹² and R¹³ furthermore together especially preferably represent C₂-C₈-alkenylene which is optionally mono- or polysubstituted by identical or different substituents from the group consisting of fluorine, chlorine, bromine, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms or represent C₃-C₈-alkylene, where the alkylene chain may in each case be interrupted by —O—, —S— or —NR¹⁸—, and where the radical is optionally mono- or polysubstituted in the alkylene moiety by identical or different substituents from the group consisting of fluorine, chlorine, cyano, methyl, ethyl, n-propyl, isopropyl, n-butyl, isobutyl, sec-butyl, tert-butyl, C₁-C₄-halogenoalkyl having 1 to 9 fluorine, chlorine and/or bromine atoms, methoxy, ethoxy, n-propoxy, isopropoxy, n-butoxy, isobutoxy, sec-butoxy, tert-butoxy, C₁-C₄-halogenoalkoxy having 1 to 9 fluorine, chlorine and/or bromine atoms, methylthio, ethylthio, n-propylthio, isopropylthio, n-butylthio, isobutylthio, sec-butylthio, tert-butylthio and C₁-C₄-halogenoalkylthio having 1 to 9 fluorine, chlorine and/or bromine atoms.

R¹⁴ and R¹⁵ independently of one another especially preferably represent hydrogen, —SO₂CF₃, methyl, ethyl, n-propyl, isopropyl, n-butyl, isobutyl, sec-butyl, tert-butyl, pentyl, hexyl, —CF₃, —CH₂CF₃, —(CF₂)₃CF₃, cyclopropyl, cyclopentyl, cyclohexyl, methoxymethyl, methoxyethyl, or represent phenyl or benzyl, each of which is optionally mono- to trisubstituted by identical or different substituents from the group consisting of fluorine, chlorine, bromine, methyl, trifluoromethyl, methoxy, trifluoromethoxy.

R¹⁴ and R¹⁵ furthermore together especially preferably represent —(CH₂)₄—, —(CH₂)₅—, —(CH₂)₆—, —CH₂—CH(CH₃)—CH₂—CH(CH₃)—CH₂—, —(CH₂)₂—O—(CH₂)₂—, —(CH₂)₂—S—(CH₂)₂— or —(CH₂)₂—N(R²²)—(CH₂)₂—.

R¹⁶ and R¹⁷ independently of one another especially preferably represent methyl, ethyl, n-propyl, isopropyl, n-butyl, isobutyl, sec-butyl, tert-butyl, n-hexyl, trifluoromethyl, trifluoroethyl, cyclopropyl, cyclopentyl, cyclohexyl or represent phenyl or benzyl, each of which is optionally mono- to tetrasubstituted by identical or different substituents from the group consisting of fluorine, chlorine, bromine, methyl, trifluoromethyl, methoxy and trifluoromethoxy.

R¹⁶ and R¹⁷ furthermore together especially preferably represent —(CH₂)₃—, —(CH₂)₄—, —(CH₂)₅—, —(CH₂)₆—, each of which is optionally mono- to tetrasubstituted by identical or different substituents from the group consisting of fluorine, chlorine, bromine, methyl, ethyl, methoxy, ethoxy, methylthio, ethylthio, trifluoromethyl, trifluoromethoxy and trifluoromethylthio.

R²⁰ and R²¹ independently of one another especially preferably represent hydrogen, methyl, ethyl, n-propyl, isopropyl, n-butyl, isobutyl, sec-butyl, tert-butyl, n-hexyl, trifluoromethyl, trifluoroethyl, cyclopropyl, cyclopentyl or cyclohexyl.

R²⁰ and R²¹ furthermore together especially preferably represent —(CH₂)₃—, —(CH₂)₄—, —(CH₂)₅—, —(CH₂)₂—O—(CH₂)₂— or —(CH₂)₂—S—(CH₂)₂—.

R²² especially preferably represents hydrogen, —SO₂R¹¹, represents —COR¹¹ or —CO₂R¹¹; represents C₁-C₁₆-alkyl, C₂-C₁₆-alkenyl, each of which is optionally mono- or polysubstituted by identical or different substituents from the group consisting of fluorine, chlorine, bromine, cyano, methylamino, ethylamino, di-(C₁-C₆-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₈-cycloalkyl, cyclopropylmethyl, cyclopentylmethyl, cyclohexylmethyl, cyclopropylethyl, cyclopentylethyl, cyclohexylethyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, cyano, methyl, ethyl, n-propyl, isopropyl,.nbutyl, isobutyl, sec-butyl, tert-butyl, C₁-C₄-halogenoalkyl having 1 to 9 fluorine, chlorine and/or bromine atoms, methoxy, ethoxy, n-propoxy, isopropoxy, n-butoxy, isobutoxy, sec-butoxy, tert-butoxy, C₁-C₄-halogenoalkoxy having 1 to 9 fluorine, chlorine and/or bromine atoms, methylthio, ethylthio, n-propylthio, isopropylthio, n-butylthio, isobutylthio, sec-butylthio, tert-butylthio and C₁-C₄-halogenoalkylthio having 1 to 9 fluorine, chlorine and/or bromine atoms.

Very particular preference is furthermore given to compounds of the formula (I-e) having the (R)-configuration in the 5-position of the pyrroline ring

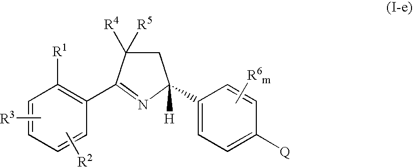

in which

R¹, R², R³, R⁴, R⁵, R⁶, m and Q have the meanings given above.

Very particular preference is furthermore given to compounds of the formula (I-f) having the (R)-configuration in the 5-position of the pyrroline ring

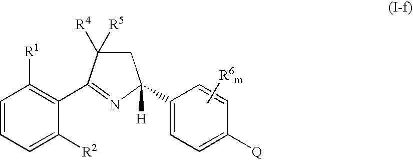

in which

R¹ represents fluorine or chlorine,

R² represents hydrogen or fluorine and

R⁴, R⁵, R⁶, m and Q have the meanings given above.

Compounds of the formulae (I-e) and (I-f) are obtained by customary processes for the optical resolution of racemates, for example by chromatography of the corresponding racemates on a chiral stationary phase. In this manner, it is possible to separate both racemic end products and racemic intermediates into the two enantiomers.

Saturated hydrocarbon radicals, such as alkyl, can in each case be straight-chain or branched, as far as this is possible, including in combination with heteroatoms, such as, for example, in alkoxy.

However, it is also possible to combine the general or preferred radical definitions or illustrations given above with one another as desired, i.e. between the respective ranges and preferred ranges. They apply both to the end products and, correspondingly, to the precursors and intermediates.

Using 5-(2,6-difluorophenyl)-2-[4′-(trifluoromethoxy)-1,1′-biphenyl-4-yl]-3,4-dihydro-2H-pyrrole and N-chlorosuccinimide as starting materials, the course of the process (A) according to the invention can be illustrated by the equation below.

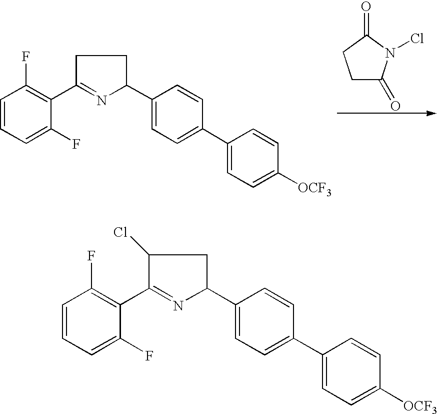

Using tert-butyl 4-(2,6-difluorophenyl)-3,3-dimethyl-4-oxo-1-[4′-(trifluoromethoxy)-1,1′-biphenyl-4-yl]-butylcarbamate as starting material and trifluoroacetic acid (TFA), the course of the process (B) according to the invention can be illustrated by the equation below.

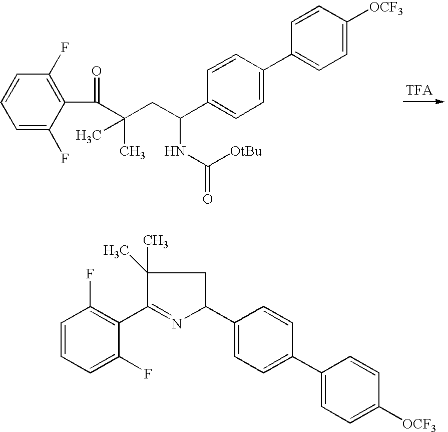

Using [2-(4-bromophenyl)cyclopropyl](2,6-difluorophenyl)-methanone, sulphuric acid and acetonitrile as starting materials, the course of the process (C) according to the invention can be illustrated by the equation below.

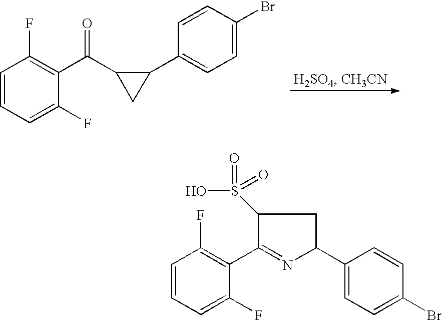

Using 5-(2,6-difluorophenyl)-2-[4′-(trifluoromethoxy)-1,1′-biphenyl-4-yl]-3,4dihydro-2H-pyrrole and 4-bromobenzaldehyde as starting materials, the course of the process (D) according to the invention can be illustrated by the equation below.

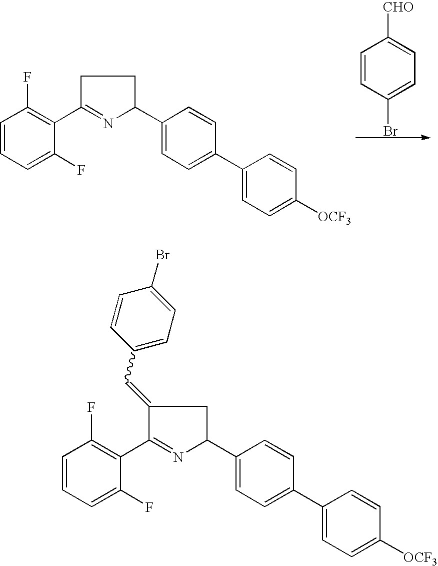

### Explanation of the Processes and Intermediates

### Process (A)

The formulae (II) and (II-a) provide general definitions of the Δ¹-pyrrolines required as starting materials for carrying out the process (A) according to the invention. In these formulae, R¹, R², R³, R⁶, m and Q preferably, particularly preferably, very particularly preferably and especially preferably have those meanings which have already been mentioned in connection with the description of the compounds of the formula (I) according to the invention as being preferred, particularly preferred, etc., for these radicals.

Pyrrolines of the formulae (II) and (II-a) are known and/or can be prepared by known processes (cf., for example, WO 98/22438, WO 99/59967 and WO 99/59968).

The formula (III) provides a general definition of the succinimides required as starting materials for carrying out the process (A) according to the invention. In this formula, Hal¹ preferably represents chlorine or bromine.

Succinimides of the formula (III) are known.

The formula (IV) provides a general definition of the sulphonyl chlorides required as starting materials for carrying out the process (A) according to the invention. In this formula, Hal² preferably represents chlorine or bromine, particularly preferably chlorine. R²³ preferably represents C₁-C₄-alkyl or C₁-C₄-halogenoalkyl, particularly preferably methyl or trifluoromethyl.

Sulphonyl chlorides of the formula (IV) are known.

Suitable diluents for carrying the process (A) according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decaline; chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole. Particular preference is given to using chloroform, carbon tetrachloride or toluene.

Suitable acid binders for carrying out the process (A) according to the invention are in each case all inorganic and organic bases customary for such reactions. Preference is given to using alkaline earth metal or alkali metal hydroxides, such as sodium hydroxide, calcium hydroxide, potassium hydroxide, or else ammonium hydroxide, alkali metal carbonates, such as sodium carbonate, potassium carbonate, potassium bicarbonate, sodium bicarbonate, and also alkali metal or alkaline earth metal acetates, such as sodium acetate, potassium acetate, calcium acetate, and alkali metal fluorides. Particular preference is given to using sodium hydroxide or potassium hydroxide.

When carrying out the process (A) according to the invention, the reaction temperatures can in each case be varied within a relatively wide range. In general, the process is carried out at temperatures between 20° C. and 140° C., preferably between 50° C. and 100° C.

When carrying out the process (A1) according to the invention, in general 1 mol or a slight excess of a compound of the formula (III) is employed per mole of the compound of the formula (II) in order to obtain the monohalogenated product. When carrying out the process (A2) according to the invention, in general 1 mol or a slight excess of a compound of the formula (IV) is employed per mole of the compound of the formula (II-a). To obtain the dihalogenated product, in general a 10-fold excess of the compound of the formula (III) is employed per mole of the compound of the formula (II). However, it is also possible to employ the reaction components in other ratios. Work-up is carried out by customary methods. In general, if appropriate after addition of a diluent, a precipitate is obtained which is filtered off. The filtrate is washed, dried, filtered and concentrated. The residue is, if appropriate, freed from any impurities that may still be present using customary methods, such as chromatography or recrystallization.

### Process (B)

The formula (V) provides a general definition of the aminoketones required as starting materials for carrying out the process (B) according to the invention. In this formula, R¹, R², R³, R⁶, m and Q preferably, particularly preferably, very particularly preferably or especially preferably have those meanings which have already been mentioned in connection with the description of the compounds of the formula (I) according to the invention as being preferred, particularly preferred, etc., for these radicals. R⁴⁻² preferably represents C₁-C₁₀-alkyl, C₁-C₁₀-halogenoalkyl, particularly preferably C₁-C₆-alkyl, C₁-C₆-halogenoalkyl have 1 to 13 fluorine, chlorine and/or bromine atom, very particularly preferably methyl, ethyl, n-propyl, isopropyl, n-butyl, isobutyl, sec-butyl, tert-butyl, trifluoromethyl or trifluoroethyl. R⁵⁻² preferably represents hydrogen, C₁-C₁₀-alkyl, C₁-C₁₀-halogenoalkyl, particularly preferably hydrogen, C₁-C₆-alkyl, C₁-C₆-halogenoalkyl having 1 to 13 fluorine, chlorine and/or bromine atoms, very particularly preferably hydrogen, methyl, ethyl, n-propyl, isopropyl, n-butyl, isobutyl, sec-butyl, tert-butyl, trifluoromethyl or trifluoroethyl.

Aminoketones of the formula (V) are novel. They can be prepared by

a) reacting lactams of the formula (IX)

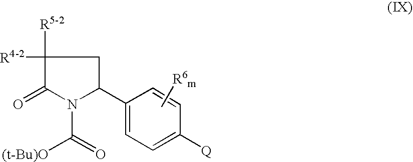

in which

R⁴⁻², R⁵⁻², R⁶, m and Q have the meanings given above, with metallated aromatic compounds of the formula (X)

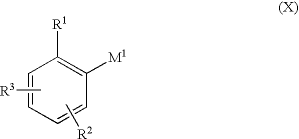

in which

R¹, R², and R³ have the meanings given above and

M¹ represents Li, MgCl, MgBr, MgI or ZnCl, at temperatures between −70° C. and +70° C., if appropriate in the presence of a diluent (for example tetrahydrofuran).

The formula (IX) provides a general definition of the lactams required as starting materials for carrying out the process (a). In this formula, R⁶, m and Q preferably, particularly preferably, very particularly preferably or especially preferably have those meanings which have already been mentioned in connection with the description of the compounds of the formula (I) according to the invention as being preferred, particularly preferred, etc., for these radicals. R⁴⁻² and R⁵⁻² preferably, particularly preferably or very particularly preferably have those meanings which have already been mentioned in connection with the description of the compounds of the formula (V) as being preferred, particularly preferred, etc., for these radicals.

Lactams of the formula (IX) are novel. They can be prepared by

b) treating pyrrolidones of the formula (XI)

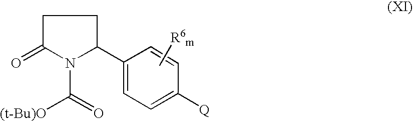

in which

R⁶, m and Q have the meanings given above, with a base (for example lithium diisopropylamide, lithium hexamethyldisilylamide) and then reacting the product with an electrophile of the formula (XII)

R⁴⁻²-G (XII)

in which

R⁴⁻² has the meanings given above and

G represents a leaving group, if appropriate in the presence of a diluent (for example tetrahydrofuran) at temperatures between −78° C. and +60° C. (cf. Tetrahedron 1999, 55 13321; Org. Lett. 199, 1, 2105).

The formula (X) provides a general definition of the metallated aromatic compounds required as starting materials for carrying out the process (a). In this formula, R¹, R² and R³ preferably, particularly preferably, very particularly preferably or especially preferably have those meanings which have already been mentioned in connection with the description of the compounds of the formula (I) according to the invention as being preferred, particularly preferred, etc., for these radicals. M¹ preferably represents Li, MgCl, MgBr, MgI or ZnCl, particularly preferably Li, MgCl, MgBr or MgI, very particularly preferably Li, MgCl or MgBr.

Metallated aromatic compounds of the formula (X) are known or can be prepared by known methods (for example lithiation or Grignard reaction) from the corresponding aromatic or halogenated aromatic compounds.

The formula (XI) provides a general definition of the pyrrolidones required as starting materials for carrying out the process (b). In this formula, R⁶, m and Q preferably, particularly preferably, very particularly preferably or especially preferably have those meanings which have already been mentioned in connection with the description of the compounds of the formula (I) according to the invention as being preferred, particularly preferred, etc., for these radicals.

Pyrrolidones of the formula (XI) are known and/or can be prepared by known processes (cf. WO 98/22438).

The formula (XII) provides a general definition of the electrophiles required as starting materials for carrying out the process (b). In this formula, R⁴⁻² preferably, particularly preferably or very particularly preferably represents those radicals which have already been mentioned in connection with the description of the aminoketones of the formula (V) as being preferred, particularly preferred, etc., for these radicals. G preferably represents halogen, methanesulphonyl, trifluoromethylsulphonyl or toluenesulphonyl.

Electrophiles of the formula (XII) are known.

If, when carrying out the process (b), one equivalent or slightly less of base and 1 equivalent of electrophile are used, what is obtained are predominantly monosubstituted compounds of the formula (IX) (where R⁵⁻²=H). If more than two equivalents of electrophile are used, what is obtained are predominantly disubstituted compounds of the formula (IX) (where R⁵⁻²≠H).

Suitable diluents for carrying out the process (B) according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decaline; chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulphoxides, such as dimethyl sulphoxide, or sulphones, such as sulpholane. Particular preference is given to using methylene chloride, chloroform, toluene, methanol or ethanol.

Suitable for carrying out the process (B) according to the invention are in each case all customary Lewis acids or protonic acids. Methods for removing the Boc group are generally known (cf, for example, T. W. Greene, P. G. M. Wuts, Protective Groups in Organic Synthesis, Ed. 3, New York, Wiley & Sons, 1999, pp. 520-525). Preference is given to using trifluoroacetic acid, HCl or HBr for removing the Boc protective group.

When carrying but the process (B) according to the invention, the reaction temperatures can in each case be varied within a relatively wide range. In general, the process is carried out at temperatures between −20° C. and +120° C., preferably between −10° C. and +60° C.

When carrying out the process (B) according to the invention, in general 100 mol of a protonic acid are employed per mole of the compound of the formula (V). However, it is also possible to employ the reaction components in other ratios. Work-up is carried out by customary methods. In general, the reaction mixture is concentrated, taken up in a suitable solvent and adjusted to pH 12 using sodium hydroxide, and the organic phase is washed with water, dried over sodium sulphate, filtered and concentrated. If appropriate, the residue is freed from any impurities that may still be present by customary methods, such as chromatography or recrystallization.

### Process (C)

The formula (VI) provides a general definition of the cyclopropanes required as starting materials for carrying out the process (C) according to the invention. In this formula, R¹, R², R³, R⁶, m and Q preferably, particularly preferably, very particularly preferably or especially preferably have those meanings which have already been mentioned in connection with the description of the compounds of the formula (I) according to the invention as being preferred, particularly preferred, etc., for these radicals.

Some of the cyclopropanes of the formula (VI) are known. They can be prepared by

c) reacting α,β-unsaturated ketones of the formula (XIII)

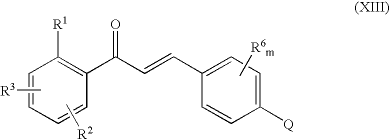

in which

R¹, R², R³, R⁶, m and Q have the meanings given above, with a trialkylsulphoxonium halide (for example trimethylsulphoxonium iodide) in the presence of a base (for example sodium hydride) and, if appropriate, in the presence of a diluent (for example dimethyl sulphoxide) (cf. Tetrahedron Asymmetry 1998 9, 1035).

The formula (VII) provides a general definition of the alcohols required as starting materials for carrying out the process (C) according to the invention. In this formula, R⁸ preferably, particularly preferably, very particularly preferably or especially preferably has those meanings which have already been mentioned in connection with the description of the compounds of the formula (I) according to the invention as being preferred, particularly preferred, etc., for these radicals.

Alcohols of the formula (VII) are known.

The formula (XIII) provides a general definition of the α,β-unsaturated ketones required as starting materials for carrying out the process (c). In this formula, R¹, R², R³, R⁶, m and Q preferably, particularly preferably, very particularly preferably or especially preferably have those meanings which have already been mentioned in connection with the description of the compounds of the formula (I) according to the invention as being preferred, particularly preferred, etc., for these radicals. α,β-Unsaturated ketones of the formula (XIII) are known and/or can be prepared by known methods.

When carrying out the process (C) according to the invention, the reaction temperatures can be varied within a relatively wide range. In general, the process is carried out at temperatures between 40° C. and 200° C., preferably between 40° C. and 140° C., particularly preferably between 6⁰° C. and 120° C.

If the process is carried out at temperatures below 40° C., what is obtained are predominantly β-ketosulphonic acid derivatives of the formula (XIV)

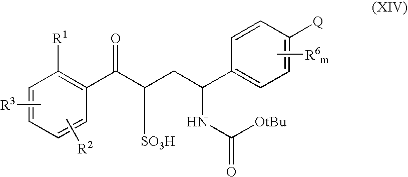

in which

R¹, R², R³, R⁶, m and Q have the meanings given above.

β-Ketosulphonic acid derivatives can be deacylated in the presence of an acid (for example 6N hydrochloric acid), if appropriate in the presence of a diluent (for example ethanol), and they then cyclize in situ to give Δ¹-pyrrolines of the formula (I-g)

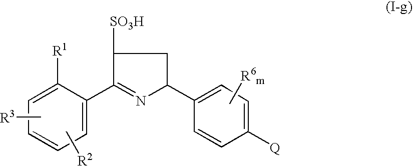

in which

R¹, R², R³, R⁶, m and Q have the meanings given above.

Δ¹-Pyrrolines of the formula (I-g) can be converted by customary methods into compounds of the formula (I-h)

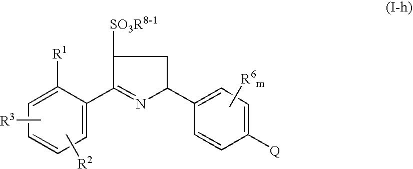

in which

R¹, R², R³, R⁶, m and Q have the meanings given above and

R⁸⁻¹ represents optionally substituted alkyl.

When carrying out the process (C) according to the invention, in general 1 ml of acetonitrile and from 1 to 10 mol of sulphuric acid are employed per mole of the compound of the formula (VI). However, it is also possible to employ the reaction components in other ratios. Work-up is carried out by customary methods. In general, the reaction mixture is diluted with water and the precipitate is filtered off with suction. If appropriate, the product is freed from any impurities that may still be present using customary methods, such as chromatography or recrystallization.

### Process (D)

The Δ¹-pyrrolines of the formula (II) required as starting materials for carrying out the process (D) according to the invention have already been described in connection with the explanation of the process (A) according to the invention.

The formula (VIII) provides a general definition of the aldehydes required as starting materials for carrying out the process (D) according to the invention. In this formula, R⁹ preferably, particularly preferably, very particularly preferably or especially preferably has those meanings which have already been mentioned in connection with the description of the compounds of the formula (I) according to the invention as being preferred, particularly preferred, etc., for this radical.

Aldehydes of the formula (VIII) are known.

Suitable diluents for carrying out the process (D) according to the invention are in each case mixtures of glacial acetic acid and alcohols, such as methanol, ethanol or propanol. Preference is given to using mixtures of glacial acetic acid and methanol.

Suitable acid binders for carrying out the process (D) according to the invention are all inorganic and organic bases which are customary for such reactions. Preference is given to using alkaline earth metal or alkali metal hydroxides, such as sodium hydroxide, calcium hydroxide, potassium hydroxide, or else ammonium hydroxide, alkali metal carbonates, such as sodium carbonate, potassium carbonate, potassium bicarbonate, sodium bicarbonate, alkali metal or alkaline earth metal acetates, such as sodium acetate, potassium acetate, calcium acetate, and also tertiary amines, such as trimethylamine, triethylamine, tributylamine, diethylisopropylamine, N,N-dimethylaniline, pyridine, N-methylpiperidine, N,N-dimethylaminopyridine, diazabicyclooctane (DABCO), diazabicyclononene (DBN) or diazabicycloundecene (DBU). Particular preference is given to using sodium acetate.

When carrying out the process (D) according to the invention, the reaction temperatures can in each case be varied within a relatively wide range. In general, the process is carried out at temperatures between 0° C. and 180° C., preferably between 20° C. and 160° C., particularly preferably between 60° C. and 1 20° C.

When carrying out the process (D) according to the invention, in general 1 mol or a slight excess of the compound of the formula (VIII) is employed per mole of the compound of the formula (II). However, it is also possible to employ the reaction components in other ratios. Work-up is carried out by customary methods. In general, the reaction mixture is concentrated and the residue is, if appropriate, freed from any impurities that may still be present using customary methods such as chromatography or recrystallization.

### Chiral Compounds of the Formulae (I-e) and (I-f)

To prepare chiral compounds of the formulae (I-e) and (I-f), it is possible, for example, to subject Δ¹-pyrrolines of the formula (II)

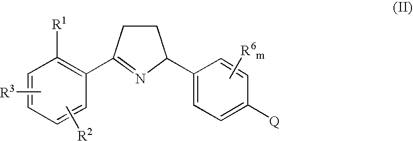

in which

R¹, R², R³, R⁶, m and Q have the meanings given above, to an optical resolution. To this end, for example, methods of preparative chromatography, preferably the High Performance Liquid Chromatography (HPLC) method, are employed. Here, a chiral stationary silica gel phase is used. A silica gel modified by tris(3,5-dimethylphenylcarbamate)-cellulose has been found to be particularly suitable for separating the compounds of the formula (II) into the two enantiomers. This separating material is commercially available. However, it is also possible to use other stationary phases. Suitable mobile phases are all customary inert organic solvents, and mixtures of these. Preference is given to using optionally halogenated aliphatic, alicyclic or aromatic hydrocarbons, such as petroleum ether, hexane, heptane, cyclohexane, dichloromethane, chloroform; alcohols, such as methanol, ethanol, propanol; nitriles, such as acetonitrile; esters, such as methyl acetate or ethyl acetate. Particular preference is given to using aliphatic hydrocarbons, such as hexane or heptane, and alcohols, such as methanol or propanol, very particularly preferably n-heptane and isopropanol or mixtures of these. In general, the separation is carried out at temperatures between 10° C. and 60° C., preferably between 10° C. and 40° C., particularly preferably at room temperature. The (R)-configured enantiomers obtained in this manner are then used as starting materials, for example in one of the processes (A) or (D) according to the invention.

All processes according to the invention are generally carried out under atmospheric pressure. However, in each case it is also possible to operate under elevated or reduced pressure.

The active compounds according to the invention are suitable for controlling animal pests, in particular insects, arachnids and nematodes, which are encountered in agriculture, in forestry, in the protection of stored products and of materials, and in the hygiene sector, and have good plant tolerance and favourable toxicity to warm-blooded animals. They may be preferably employed as plant protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

In particular, the compounds of the formula (I) according to the invention have excellent activity against caterpillars, beetle larvae, spider mites, aphids and leaf-mining flies.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

The active compounds according to the invention can be converted into the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds according to the invention with extenders, that is liquid solvents and/or solid carriers, optionally with the use of surfactants, that is emulsifiers and/or dispersants and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

As solid carriers there are suitable:

for example ammonium salts and ground natural minerals such as kaolins, clays, talc, chalk, quartz, attapulgite, montmorillonite or diatomaceous earth, and ground synthetic minerals, such as highly disperse silica, alumina and silicates; as solid carriers for granules there are suitable: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks;

as emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates;

as dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be present in their commercially available formulations and in the use forms prepared from these formulations as a mixture with other active compounds, such as insecticides, attractants, sterilizing agents, bactericides, acaricides, nematicides, fungicides, growth-regulating substances or herbicides. The insecticides include, for example, phosphoric acid esters, carbamates, carboxylates, chlorinated hydrocarbons, phenylureas and substances produced by microorganisms, inter alia.

Particularly advantageous co-components are, for example, the following:

### Fungicides

aldimorph, ampropylfos, ampropylfos-potassium, andoprim, anilazine, azaconazole, azoxystrobin,

benalaxyl, benodanil, benomyl, benzamacril, benzamacryl-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

ediphenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur,

quinconazole, quintozene (PCNB),

sulphur and sulphur preparations,

tebuconazole, tecloftalam, tecnazene, tetcyclasis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforine, triticonazole,

uniconazole,

validamycin A, vinclozolin, viniconazole,

zarilamide, zineb, ziram and also

Dagger G, OK-8705, OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-isopropyl{2-methyl-1-[[[1-(4-methylphenyl)-ethyl]-amino]-carbonyl]-propyl}-carbamate,

1-(2,4-dichlorophenyl)-2-(1H-1,2,4-triazol-1-yl)-ethanone-O-(phenylmethyl)-oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinole,

2′, 6′-dibromo-2-methyl-4′-trifluoromethoxy-4′-trifluoro-methyl-1,3-thiazole-5-carboxanilide,

2,2-dichloro-N-[1-(4-chlorophenyl)-ethyl]-1-ethyl-3-methyl-cyclopropanecarboxamide,

2,6-dichloro-5-(methylthio)-4-pyrimidinyl-thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-α-D-glycopyranosyl]-α-D-glucopyranosyl]-amino]4methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano[(1-methyl-2-propynyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-(1,1-dimethylethyl)-N-ethyl-N-propyl-1,4-dioxaspiro[4.5]decane-2-methanamine,

8-hydroxyquinoline sulphate,

9H-xanthene-2-[(phenylamino)-carbonyl]-9-carboxylic hydrazide,

bis-(1-methylethyl)-3-methyl-4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholine hydrochloride,

ethyl[(4-chlorophenyl)-azo]-cyanoacetate,

potassium bicarbonate,

methanetetrathiol-sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,3-dichloro-4-hydroxyphenyl)-1-methyl-cyclohexanecarboxamide,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrimidinamine,

N-(4-hexylphenyl)-1,4,5,6-tetrahydro-2-pyrimidinamine,

N-(5-chloro-2-methylphenyl)-2-methoxy-N-(2-oxo-3-oxazolidinyl)-acetamide,

N-(6-methoxy-3-pyridinyl)-cyclopropanecarboxamide,

N-[2,2,2-trichloro-1-[(chloroacetyl)-amino]-ethyl]-benzamide,

N-[3-chloro-4,5-bis-(2-propinyloxy)-phenyl]-N′-methoxy-methanimidamide,

N-formyl-N-hydroxy-DL-alanine-sodium salt,

O,O-diethyl[2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′(3′H)-isobenzofuran]-3′-one,

### Bactericides

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, BPMC, bromophos A, bufencarb, buprofezin, butathiofos, butocarboxim, butylpyridaben,

cadusafos, carbaryl, carbofuran, carbophenothion, carbosulfan, cartap, chloetbocarb, chlorethoxyfos, chlorfenapyr, chlorfenvinphos, chlorfluazuron, chlormephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine,

deltamethrin, demeton M, demeton S, demeton—S-methyl, diafenthiuron, diazinon, dichlorvos, diflubenzuron, dimethoate, dimethylvinphos, diofenolan, disulfoton, docusat-sodium, dofenapyn,

eflusilanate, emamectin, empenthrin, endosulfan, Entomopfthora spp., eprinomectin esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, isazofos, isofenphos, isoxathion, ivermectin,

nuclear polyhedrosis viruses,

lambda-cyhalothrin, lufenuron,

malathion, mecarbam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, selamectin, silafluofen, spinosad, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenylmethyl)-3-furanyl]-methyl-3-[(dihydro-2-oxo-3(2H)-furanylidene)-methyl]-2,2-dimethylcyclopropanecarboxylate,

(3-phenoxyphenyl)-methyl-2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate.

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate,

It is also possible to admix other known active compounds, such as herbicides, fertilizers and growth regulators.

When used as insecticides, the active compounds according to the invention can furthermore be present in their commercially available formulations and in the use forms, prepared from these formulations, as a mixture with synergistic agents. Synergistic agents are compounds which increase the action of the active compounds according to the invention, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and parts thereof according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having specific properties (“traits”) which can be obtained by conventional breeding, by mutagenesis or by recombinant DNA techniques. This can be varieties, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferred according to the invention include all plants which, in the genetic modification, received genetic material which imparted particularly advantageous useful traits to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapevines), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexius, elicitous and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still to be developed genetic traits, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattella germanica and Supella spp.

From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.

From the order of the Actinedida (Prostigmata) und Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

They have, for example, excellent activity against the development stages of ticks such as, for example, Amblyomma hebraeum, against parasitic flies such as, for example, Lucilia cuprina and against fleas such as, for example, Ctenocephalides felis.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) according to the invention can be used as formulations (for example powders, emulsions, free-flowing compositions), which comprise the active compounds according to the invention in an amount of 1 to 80% by weight, directly or after 100 to 10 000-fold dilution, or they can be used as a chemical bath.

It has furthermore been found that the compounds according to the invention have a strong insecticidal action against insects which destroy industrial materials.

The following insects may be mentioned as examples and as preferred—but without a limitation:

Beetles, such as

Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.

Hymenopterons, such as

Sirex juvencus, Urocerus gigas, Urocerus gigas taignus, Urocerus augur.

Termites, such as

Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccarina.

Industrial materials in the present connection are to be understood as meaning non-living materials, such as, preferably, plastics, adhesives, sizes, papers and cards, leather, wood and processed wood products and coating compositions.

Wood and processed wood products are materials to be protected, very particularly preferably, from insect infestation.

Wood and processed wood products which can be protected by the agents according to the invention or mixtures comprising these are to be understood as meaning, for example:

building timber, wooden beams, railway sleepers, bridge components, boat jetties, wooden vehicles, boxes, pallets, containers, telegraph poles, wood panelling, wooden window frames and doors, plywood, chipboard, joinery or wooden products which are used quite generally in house-building or in building joinery.

The active compounds according to the invention can be used as such, in the form of concentrates or in generally customary formulations, such as powders, granules, solutions, suspensions, emulsions or pastes.

The formulations mentioned can be prepared in a manner known per se, for example by mixing the active compounds according to the invention with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., terpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably a-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, provided that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-coumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or a drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phtbalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether, or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, pernethrin, imidacloprid, NI-25, flufenoxuron, hexaflumuron, transfluthrin, thiacloprid, methoxyfenozide and triflumuron, and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-dichloro-N-octylisothiazolin-3-one.

The compounds according to the invention can at the same time be employed for protecting objects which come into contact with saltwater or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas .and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fuingicides, herbicides, molluscicides, or other antifouling active compounds.

Preferably suitable components in combinations with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as

benzo[b]thiophenecarboxylic acid cyclohexylamide S,S-dioxide, dichlofluanid, fluorfolpet, 3-iodo-2-propinyl butylcarbamate, tolylfluanid and azoles such as azaconazole, cyproconazole, epoxyconazole, hexaconazole, metconazole, propiconazole and tebuconazole;

molluscicides such as

fentin acetate, metaldehyde, methiocarb, niclosamid, thiodicarb and trimethacarb; or conventional antifouling active compounds such as 4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmaleiimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, flugicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/-styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the Theological properties and other conventional constituents. The compounds according to the invention or the abovementioned mixtures may also be incorporated into self-polishing antifouling systems.

The active compounds according to the invention are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all development stages. These pests include:

From the order of the Scorpionidea, for example, iButhus occitanus.

From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeanais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, growth regulators or active compounds from other known classes of insecticides.

They are used as aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free or passive evaporation systems, moth papers, moth bags and moth gels, as granules or dusts, in baits for spreading or in bait stations.

The preparation and use of the susbstances according to the invention is shown in the examples below.

### PREPARATION EXAMPLES

### Example 1

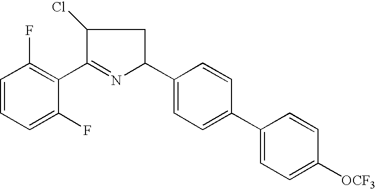

5-(2,6-Difluorophenyl)-2-[4′-(trifluoromethoxy)-1,1′-biphenyl-4-yl]-3,4-dihydro-2H-pyrrole (1.00 g, 2.40 mmol) is initially charged in chloroform (50 ml) and cooled to 0° C. At this temperature, N-chlorosuccinimide (0.33 g, 2.47 mmol) is added a little at a time. After 0.5 hours of stirring at room temperature, the mixture is heated at reflux for 5 hours. After cooling, the resulting succinimide is filtered off and the solvent is removed under reduced pressure. The product is isolated as a mixture of diastereomers.

This gives 1.02 g (91% of theory) of 4-chloro-5-(2,6-difluorophenyl)-2-[4′-(trifluoromethoxy)-1,1′-biphenyl-4-yl]-3,4-dihydro-2H-pyrrole.

HPLC: log P₁=5.31 (52.63%); log P₂ 5.39 (43.88%).

### Example 2

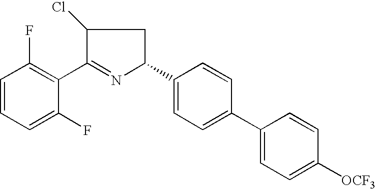

Starting from (2R)-5-(2,6-difluorophenyl)-2-[4′-(trifluoromethoxy)-1,1′-biphenyl-4yl]-3,4-dihydro-2H-pyrrole, the compound (2R)-4-chloro-5-(2,6-difluorophenyl)-2-[4′-(trifluoromethoxy)-1,1′-biphenyl-4-yl]-3,4-dihydro-2H-pyrrole is obtained analogously to Example 1.

HPLC: log P₁=5.31; log P₂=5.39.

### Example 3

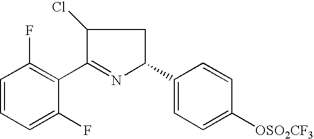

4-[(2R)-5-(2,6-Difluorophenyl)-3,4-dihydro-2H-pyrrol-2-yl]phenyl-trifluoromethane-sulphonate (1.00 g, 2.47 mmol) is initially charged in chloroform (50 ml) and cooled to 0° C. At this temperature, N-chlorosuccinimide (0.33 g, 2.47 mmol) is added a little at a time. After 0.5 hours of stirring at room temperature, the mixture is heated at reflux for 16 hours. The solvent is removed under reduced pressure and the residue is stirred with n-pentane. The precipitate is filtered off and discarded. The filtrate is concentrated. The product is obtained as a mixture of diastereomers.

This gives 1.08 g (100% of theory) of 4-[(2R)-4-chloro-5-(2,6-difluorophenyl)-3,4-dihydro-2H-pyrrol-2-yl]phenyl-trifluoromethanesulphonate.

HPLC: log P₁=4.34 (50.38%); log P₂=4.40 (49.62%).

NMR (CD₃CN): δ=2.40 (1H, m), 2.85 (1H, m), 3.37 (1H, m), 5.58 (1H, m) 7.15 (2H, m), 7.38-7.59 (5H, m) ppm.

### Example 4

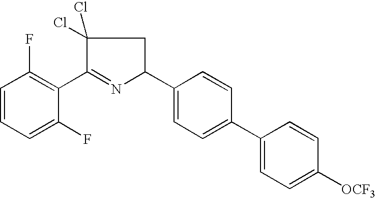

5-(2,6-Difluorophenyl)-2-[4′-(trifluoromethoxy)-1,1′-biphenyl-4-yl]-3,4-dihydro-2H-pyrrole (1.00 g, 2.40 mmol) is initially charged in carbon tetrachloride (20 ml) and cooled to 0° C. At this temperature, N-chlorosuccinimide (1.28 g, 9.60 mmol) is added a little at a time. After 0.5 hours of stirring at room temperature, the mixture is heated at reflux for S hours. The solvent is removed under reduced pressure and the crude product is purified by silica gel chromatography (mobile phase: n-hexane/ethyl acetate 4:1).

This gives 1.00 g (81% of theory) of 4,4-dichloro-5-(2,6-difluorophenyl)-2-[4′-(trifluoromethoxy)-1,1′-biphenyl-4-yl]-3,4-dihydro-2H-pyrrole.

HPLC: log P=5.92 (94.4%).

M.p.: 60-61° C.

NMR (CD₃CN): δ=2.87 (1H, dd), 3.68 (1H, dd), 5.55 (1H, dd), 7.20 (2H, m), 7.38 (2H, d), 7.46 (2H, d), 7.63 (1H, m), 7.69 (2H, d), 7.75 (2H, d) ppm.

### Example 5

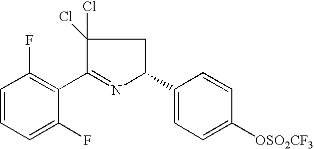

4-[(2R)-5-(2,6-Difluorophenyl)-3,4-dihydro-2H-pyrrol-2-yl]phenyl-trifluoromethane-sulphonate (1.00 g, 2.47 mmol) is initially charged in chloroform (50 ml) and cooled to 0° C. At this temperature, N-chlorosuccinimide (6.60 g, 49.4 mmol) is added a little at a time. After 0.5 hours of stirring at room temperature, the mixture is heated at reflux for 16 hours. The solvent is removed under reduced pressure and the residue is stirred with n-pentane. The precipitate is filtered off and discarded. The filtrate is concentrated.

This gives 0.73 g (61% of theory) of 4-[(2R)-4,4-dichloro-5-(2,6-difluorophenyl)-3,4dihydro-

HPLC: log P=4.88 (97.5%).

NMR (CD₃CN): δ=2.80 (1H, dd), 3.67 (1H, dd), 5.56 (1H, dd), 7.19 (2H, m), 7.42 (2H, d), 7.52 (2H, d), 7.62 (1H, m) ppm.

### Example 6

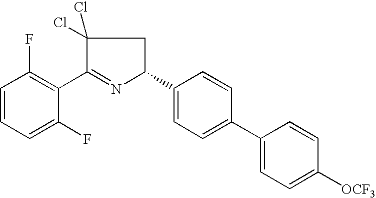

(2R)-5-(2,6-Difluorophenyl)-2-[4′-(trifluoromethoxy)-1,1′-biphenyl-4-yl]-3,4-dihydro-2H-pyrrole (0.50 g, 1.20 mmol) is initially charged in chloroform (50 ml) and cooled to 0° C. At this temperature, N-chlorosuccinimide (6.41 g, 48.0 mmol) is added a little at a time. After 0.5 hours of stirring at room temperature, the mixture is heated at reflux for 16 hours. The solvent is removed under reduced pressure and the residue is stirred with n-pentane. The precipitate is filtered off and discarded. The filtrate is concentrated.

This gives 0.54 g (89% of theory) of (2R)-4,4-dichloro-5-(2,6-difluorophenyl)-2-[4′-(trifluoromethoxy)-1,1′-biphenyl-4-yl]-3,4-dihydro-2H-pyrrole.

| | |
|---|---|
| HPLC: | log P = 5.91 (95.86%) |
| M.p.: | 76° C. |

NMR (CD₃CN): δ=2.87 (1H, dd), 3.68 (1H, dd), 5.55 (1H, dd), 7.20 (2H, m), 7.38 (2H, d), 7.46 (2H, d), 7.63 (1H, m), 7.69 (2H, d), 7.75 (2H, d) ppm.

### Example 7

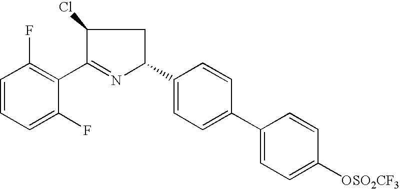

4′-[(2R)-5-(2,6-Difluorophenyl)-3,4-dihydro-2H-pyrrol-2-yl]-1,1′-biphenyl-4-ol (3.49 g, 0.01 mol) is suspended in toluene (75 ml). Aqueous sodium hydroxide solution (0.9 ml, 45% w/v) is added, and the reaction mixture is stirred at room temperature for 0.75 hours. At this temperature, trifluoromethanesulphonyl chloride (1.6 ml, 0.015 mol) is added dropwise. The mixture is stirred at room temperature for 16 hours. The precipitate is filtered off. The filtrate is washed successively with 1N aqueous sodium hydroxide solution, saturated sodium bicarbonate solution and saturated ammonium chloride solution. The organic phase is dried over sodium sulphate, filtered and concentrated. The crude product is purified by silica gel chromatography (mobile phase: n-hexane/ethyl acetate 9:1).

This gives 0.41 g (8% of theory) of 4′-[(2R)-4-chloro-5-(2,6-difluorophenyl)-3,4-dihydro-2-yl]-1,1′-biphenyl-4-yl trifluoromethanesulphonate.

HPLC: log P=5.22 (98.7%).

NMR (CD₃CN): δ=2.10 (1H, m), 3.35 (1H, m), 5.45 (2H, m), 7.15 (2H, m), 7.47-7.60 (5H, m), 7.69 (2H, d), 7.80 (2H, d) ppm.

### Example 8

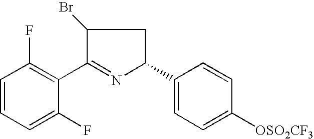

4-[(2R)-5-(2,6-Difluorophenyl)-3,4-dihydro-2H-pyrrol-2-yl]phenyl-trifluoromethane-sulphonate (1.00 g, 2.47 mmol) is initially charged in chloroform (50 ml) and cooled to 0° C. At this temperature, N-bromosuccinimide (0.44 g, 2.47 mmol) is added a little at a time. The mixture is then stirred at room temperature for 0.5 hours and then heated at reflux for 16 hours. The solvent is removed under reduced pressure and the residue is stirred with n-pentane. The precipitate is filtered off and discarded. The filtrate is concentrated. The product is obtained as a mixture of diastereomers.

This gives 0.49 g (36%. of theory) of 4-[(2R)-4-bromo-5-(2,6-difluorophenyl)-3,4-dihydro-2-pyrrol-2-yl]phenyl-trifluoromethanesulphonate.

HPLC: log P₁=4.39 (28.13%); log P₂=4.49 (58.80%).

LC-MS (ESI-positive): Rt_{min.}=7.54 [M+H]⁺ (485.9; ⁸¹Br); Rtₘᵢₙ=7.64 [M+H]⁺ (484.0; ⁷⁹Br).

### Example 9

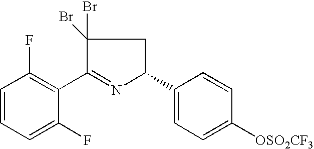

4-[(2R)-5-(2,6-Difluorophenyl)-3,4-dihydro-2H-pyrrol-2-yl]phenyl-trifluoromethane-sulphonate (1.00 g, 2.47 mmol) is initially charged in chloroform (50 ml) and cooled to 0° C. At this temperature, N-bromosuccinimide (8.80 g, 49.4 mmol) is added a little at a time. The mixture is then stirred at room temperature for 0.5 hours and then heated at reflux for 16 hours. The solvent is removed under reduced pressure and the residue is stirred with n-pentane. The precipitate is filtered off and discarded. The filtrate is concentrated. The crude product is purified by silica gel chromatography (mobile phase: n-hexane/ethyl acetate 4:1). This gives 0.26 g (18% of theory) of 4-[(2R)-4,4-dibromo-5-(2,6-difluorophenyl)-3,4-dihydro-2H-pyrrol-2-yl]phenyl-trifluoromethanesulphonate.

HPLC: log P=4.97 (95.04%).

NMR (CD₃CN): δ=3.07 (1H, dd), 3.86 (1H, dd), 5.52 (1H, dd), 7.18 (2H, m), 7.43 (2H, d),.7.65 (1H, m), 7.89 (2H, d) ppm.

### Example 10

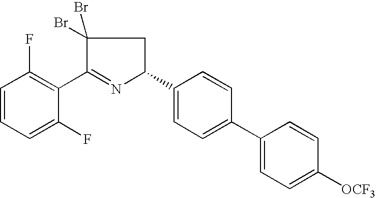

(2R)-5-(2,6-Difluorophenyl)-2-[4′-(trifluoromethoxy)-1,1′-biphenyl-4-yl]-3,4-dihydro-2H-pyrrole (1.00 g, 2.40 mmol) is initially charged in chloroform (50 ml) and cooled to 0° C. At this temperature, N-bromosuccinimide (8.54 g, 48.0 mmol) is added a little at a time. The mixture is then stirred at room temperature for 0.5 hours and then heated at reflux for 5 hours. The solvent is removed under reduced pressure and the residue is stirred with n-pentane. The precipitate is filtered off and discarded. The filtrate is concentrated. The crude product is purified by silica gel chromatography (mobile phase: n-hexane/ethyl acetate 4:1).

This gives 0.13 g (9% of theory) of (2R)-4,4-dibromo-5-(2,6-difluorophenyl)-2-[4′-(trifluoromethoxy)-1,1′-biphenyl-4-yl]-3,4-dihydro-2H-pyrrole.

HPLC: log P=6.01 (93.26%).

NMR (CD₃CN): δ=3.09 (1H, dd), 3.85 (1H, dd), 5.50 (1H, dd), 7.20 (2H, m), 7.40 (2H, d), 7.47 (2H, d), 7.61 (1H, m), 7.66 (2H, d), 7.72 (2H, d) ppm.

### Example 11

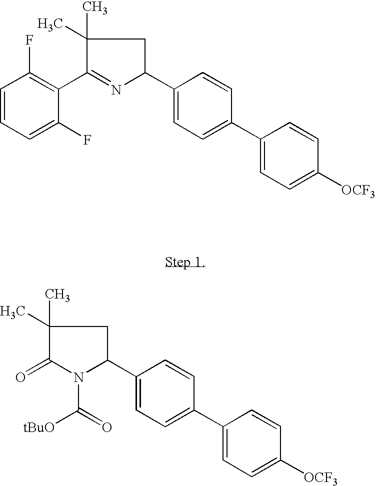

tert-Butyl 2-oxo-5-[4′-(trifluoromethoxy)-1,1′-biphenyl-4-yl]-1-pyrrolidine-carboxylate (4.21 g, 10.0 mmol) is initially charged in tetrahydrofuran (50 ml). At −78° C., lithium bis(trimethylsilylamide) (22.0 ml, 1.0 M in tetrahydrofuran, 22.0 mmol) is added dropwise, and the reaction mixture is stirred at this temperature for one hour. Methyl iodide (3.12 g, 22.0 mmol) is added dropwise at −78° C. The mixture is stirred at this temperature for two hours and then allowed to warm to room temperature over a period of 16 hours. The mixture is poured into an aqueous ammonium chloride solution and extracted with ethyl acetate. The organic phase is washed with water, dried over magnesium sulphate, filtered and concentrated. The crude product is purified by silica gel chromatography (mobile phase: cyclohexane/ethyl acetate 8:1).

This gives 1.84 g (40% of theory) of tert-butyl 3,3-dimethyl-2-oxo-5-[4′-(trifluoromethoxy)-1,1′-biphenyl-4-yl]-1-pyrrolidinecarboxylate.

HPLC: log P=4.99 (98.2%).

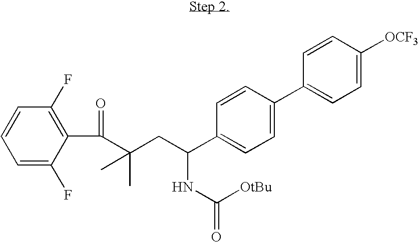

1,3-Difluorobenzene (0.48 g, 4.18 mmol) is initially charged in tetrahydrofuran (25 ml). At −78° C., n-butyllithium (2.61 ml, 1.6 M in hexane, 4.18 mmol) is added dropwise, and the mixture is stirred at this temperature for one hour. A solution of tert-butyl 3,3-dimethyl-2-oxo-5-[4′-(trifluoromethoxy)-1,1′-biphenyl-4-yl]-1-pyrrolidine-carboxylate (1.70 g, 3.8 mmol) in tetrahydrofuran (50 ml) is added at −78° C. The mixture is stirred at this temperature for two hours and then allowed to warm to room temperature over a period of 16 hours. The mixture is poured into water and extracted with ethyl acetate. The organic phase is washed with water, dried over magnesium sulphate, filtered and concentrated. The crude product is purified by silica gel chromatography (mobile phase: cyclohexane/ethyl acetate 20:1→10:1).

This gives 0.63 g (19% of theory) of tert-butyl 4-(2,6-difluorophenyl)-3,3-dimethyl-4-oxo-1-[4′-(trifluoromethoxy)-1,1′-biphenyl-4-yl]butylcarbamate.

HPLC: log P=6.45 (65.4%).

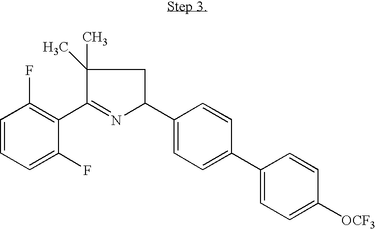

tert-Butyl 4-(2,6-difluorophenyl)-3,3-dimethyl-4-oxo-1-[4′-(trifluoromethoxy)-1,1′-biphenyl-4-yl]butylcarbamate (0.50 g, 0.89 mmol) is initially charged in dichloromethane (10 ml) and cooled to 0° C. Trifluoroacetic acid (0.68 ml, 8.9 mmol) is added dropwise. The mixture is stirred at room temperature for 3 hours and evaporated to dryness. The residue is adjusted to pH 11 using 2M aqueous sodium hydroxide solution and then extracted with ethyl acetate. The organic phase is washed with water, dried over magnesium sulphate, filtered and concentrated.

This gives 0.19 g (46% of theory) of 4,4-dimethyl-5-(2,6-difluorophenyl)-2-[4′-(trifluoromethoxy)-1,1′-biphenyl-4-yl]-3,4-dihydro-2H-pyrrole.

HPLC: log P=5.67 (95.6%).

NMR (CD₃CN): δ=1.17 (3H, s), 1.25 (3H, m), 1.84 (1H, dd), 2.56 (1H, dd), 5.33 (1H, m), 7.09 (2H, m), 7.39 (2H, d), 7.43 (2H, d), 7.50 (1H, m), 7.64 (2H, d), 7.72 (2H, d) ppm.

### Example 12

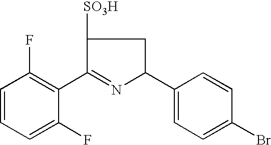

Sulphuric acid (4.90 g, 98% strength, 50.0 mmol) is initially charged at 0° C. Acetonitrile (12.5 ml) is added dropwise, and the mixture is stirred at 0° C. for 0.5 hours. The reaction mixture is cooled to −10° C., and a solution of [2-(4-bromophenyl)cyclopropyl](2,6-difluorophenyl)methanone (4.21 g, 12.50 mmol) in acetonitrile (47.5 ml) is added dropwise. The mixture is allowed to warm slowly to room temperature and then stirred at 80° C. for 16 hours. The reaction mixture is cooled to room temperature and diluted with water, and the precipitate is filtered off with suction. The product is obtained as a mixture of diastereomers.

This gives 1.30 g (24% of theory) of 2-(4-bromophenyl)-5-(2,6-difluorophenyl)-3,4-dihydro-2H-pyrrole-4-sulphonic acid.

HPLC: log P₁=1.40 (32%); log P₂=1.60 (64.8%).

LC-MS (ESI-positive): Rtₘᵢₙ=3.34 [M+H]⁺ (417.9); Rtₘᵢₙ=3.69 [M+H]⁺ (417.9).

NMR (DMSO): δ=2.35 (1H, m), 2.42 (1H, m), 3.05 (1H, m), 3.17 (1H, m), 4.87 (1H, m), 4.94 (1H, m), 5.60 (1H, m), 5.68 (1H, m), 7.29 (4H, m), 7.48 (2H, d), 7.54 (2H, d), 7.61 (2H, d), 7.67 (2H, d), 7.73 (2H, m) ppm.

### Example 13

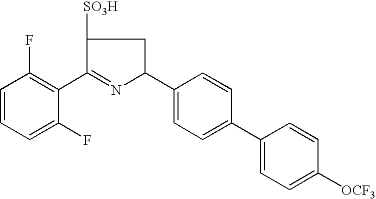

Sulphuric acid (3.65 g, 98% strength, 37.3 mmol) is initially charged at 5° C. Acetonitrile (9.3 ml) is added dropwise, and the mixture is stirred at 0° C. for 0.5 hours. The reaction mixture is cooled to −10° C., and a solution of (2,6-difluorophenyl){2-[4′-(trifluoromethoxy)-1,1′-biphenyl-4-yl]cyclopropyl}methanone (3.89 g, 9.3 mmol) in acetonitrile (27 ml) is added dropwise. The mixture is allowed to warm slowly to room temperature and stirred at this temperature for 48 hours. The reaction mixture is then stirred at 80° C. for 16 hours. The mixture is cooled to room temperature and diluted with water (50 ml), and the precipitate is filtered off with suction. The product is obtained as a mixture of diastereomers.

This gives 3.80 g (68% of theory, moist product) of 5-(2,6-difluorophenyl)-2-[4′-(trifluoromethoxy)-1,1′-biphenyl-4-yl]-3,4-dihydro-2H-pyrrole-4-sulphonic acid.

HPLC: log P₁=2.32 (27.5%); log P₂=2.46 (55.2%).

LC-MS (ESI-positive): Rtₘᵢₙ=4.99 [M+H]⁺ (498.1); Rtₘᵢₙ=5.19 [M+H]⁺ (498.1).

The residue is stirred with ethyl acetate and filtered off with suction. The product (second batch) is obtained as a mixture of diastereomers.

This gives 2.80 g (59% of theory) of 5-(2,6-difluorophenyl)-2-[4′-(trifluoromethoxy)-1,1′-biphenyl-4-yl]-3,4-dihydro-2H-pyrrole4-sulphonic acid.

HPLC: log P₁=2.29 (32.2%); log P₂=2.46 (66.1%).

NMR (DMSO): δ=2.35 (1H, m), 2.99-3.17 (1H, m), 4.72-4.80 (1H, m), 5.52-5.65 (1H, m), 7.23 (2H, m), 7.46 (2H, m), 7.54 (1H, d), 7.63-7.84 (6H, m) ppm.

### Example 14

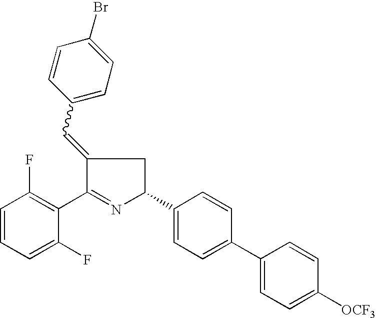

(2R)-5-(2,6-Difluorophenyl)-2-[4′-(trifluoromethoxy)-1,1′-biphenyl-4-yl]-3,4-dihydro-2H-pyrrole (1.00 g, 2.40 mmol), 4-bromobenzaldehyde (0.89 g, 4.80 mmol), sodium acetate (0.39 g, 4.80 mmol) and glacial acetic acid (0.21 g, 4.80 mmol) are heated in methanol (20 ml) at reflux for 48 hours. The mixture is evaporated to dryness and the crude product is purified by silica gel chromatography (mobile phase: n-hexane/ethyl acetate 10:1).

This gives 0.34 g (24% of theory) of (2R)-4-(4-bromobenzylidene)-5-(2,6-difluorophenyl)-2-[4′-(trifluoromethoxy)-1,1′-biphenyl-4-yl]-3,4-dihydro-2H-pyrrole.

HPLC: log P=6.43 (100%).

NMR (DMSO): δ=2.91 (1H, m), 3.68 (1H, m), 5.69. (1H, m), 6.49 (1H, s), 7.32 (2H, m), 7.40-7.49 (6H, m), 7.56 (2H, d), 7.70 (3H, m), 7.79 (2H, d) ppm.

### Example 15

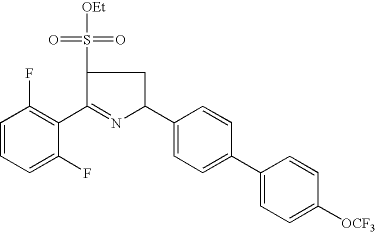

5-(2,6-Difluorophenyl)-2-[4′-(trifluoromethoxy)-1,1′-biphenyl-4-yl]-3,4-dihydro-2H-pyrrole-4-sulphonic acid (Ex. 13) (0.32 g, 0.643 mmol) and ethyl orthoformate (0.953 g, 6.433 mmol) are combined and then allowed to react further at 100° C. for 20 hours. The reaction mixture is taken up in 50 ml of ethyl acetate and washed twice with in each case 30 ml of water, and the organic phase is dried over sodium sulphate, filtered and concentrated under reduced pressure. The crude product is purified by silica gel chromatography (mobile phase: n-hexane/ethyl acetate 4:1).

This gives 0.12 g (28% of theory) of ethyl 5-(2,6-difluorophenyl)-2-[4′-(trifluoromethoxy)-1,1′-biphenyl-4-yl]-3,4-dihydro-2H-pyrrole-4-sulphonate.

HPLC: log P=4.77 (36.5%).

NMR (DMSO): δ=1.2 (3H, m), 4.2 (2H, m), 5.6 (1H, m), 7.0-7.9 (11H, m) ppm.

### Example 16

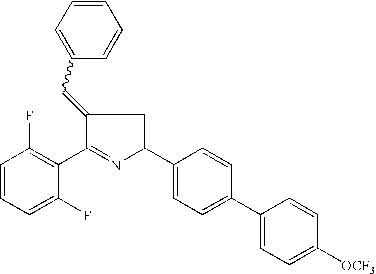

5-(2,6-Difluorophenyl)-2-[4′-(trifluoromethoxy)-1,1′-biphenyl-4-yl]-3,4-dihydro-2H-pyrrole (2.00 g, 4.79 mmol) and benzaldehyde (1.02 g, 9.58 mmol) are initially charged in 40 ml of methanol. Sodium acetate (0.79 g, 9.58 mmol) and then glacial acetic acid (0.58 g, 9.58 mmol) are then added, and the mixture is stirred at room temperature for 4 days. The reaction mixture is then allowed to cool and concentrated. The residue is taken up in ethyl acetate and extracted successively with saturated sodium bicarbonate solution and water. The organic phase is separated off, dried over magnesium sulphate, filtered and concentrated. The crude product is purified by silica gel chromatography (mobile phase: cyclohexane→cyclohexane/ethyl acetate 10:1).

This gives 1.38 g (55% of theory) of 4-benzylidene-5-(2,6-difluorophenyl)-2-[4′-(trifluoromethoxy)-1,1′-biphenyl-4-yl]-3,4-dihydro-2H-pyrrole.

HPLC: log P=5.77 (97%).

NMR (CD₃CN): δ=2.9-3.0 (1H, m), 3.63-3.70 (1H, m), 5.63-5.65 (1H, m), 7.15-7.19 (2H, m), 7.33-7.48 (9H, m), 7.64 (2H, m), 7.66 (2H, m) ppm.

### Example 17

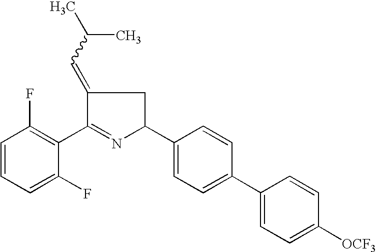

5-(2,6-Difluorophenyl)-2-[4′-(trifluoromethoxy)-1,1′-biphenyl-4-yl]-3,4-dihydro-2H-pyrrole (2.00 g, 4.78 mmol) and 2-methylpropionaldehyde (0.69 g, 9.58 mmol) are initially charged in 40 ml of methanol. Sodium acetate (0.79 g, 9.58 mmol) and then glacial acetic acid (0.58 g, 9.58 mmol) are then added, and the mixture is stirred at room temperature and under argon for 4 days. The reaction mixture is then allowed to cool and concentrated. The residue is taken up in ethyl acetate and extracted successively with saturated sodium carbonate solution and water. The organic phase is separated off, dried over magnesium sulphate, filtered and concentrated. The crude product is purified by silica gel chromatography (mobile phase: cyclohexane→cyclohexane/ethyl acetate 20:1).

This gives 0.1 g (3.7% of theory) of 4-(2-methylpropylidene)-5-(2,6-difluorophenyl)-2-[4′-(trifluoromethoxy)-1,1′-biphenyl-4-yl]-3,4-dihydro-2H-pyrrole.

HPLC: log P=5.80 (84%).

NMR (CD₃CN): δ=0.94-1.00 (6H, m), 2.50 (1H, m), 2.60 (1H, m), 3.30 (1H, m), 5.50 (1H, m), 7.10 (2H, in), 7.40 (4H, m), 7.50 (1H, m), 7.60 (2H, m), 7.70 (2H, m) ppm.

The stated logP values were determined in accordance with EEC directive 79/831 Annex V.A8 by HPLC (High Performance Liquid Chromatography) using a reversed-phase column (C 18). Temperature: 43° C.

In the acidic range, the determination was carried out at pH 2.3 using the mobile phases 0.1% aqueous phosphoric acid and acetonitrile; linear gradient from 10% acetonitrile to 90% acetonitrile.

Calibration was carried out using unbranched alkan-2-ones (having 3 to 16 carbon atoms) with known logP values (determination of the logP values using the retention times, by linear interpolation of two successive alkanones).

The lambda max values were determined in the maxima of the chromatographic signals using the UV spectra from 200 nm to 400 nm.

### Use Examples

### Example A

### Heliothis virescens Test

Solvent: 30 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Soyabean shoots (Glycine max) are treated by being dipped into the preparation of active compound of the desired concentration and populated with Heliothis virescens caterpillars whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE A
Plant-damaging insects
Heliothis virescens test

| | Active compound | |
|---|---|---|
| | concentration in | Kill rate in % |
| Active compounds | ppm | after 6^{d} |
| 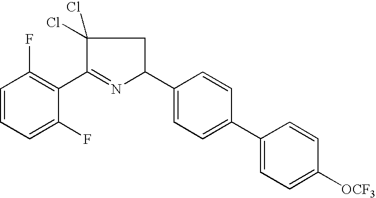 | 1000 | 100 |
| 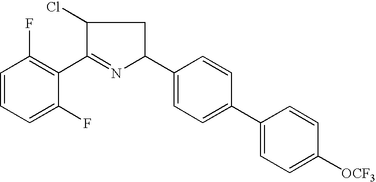 | 1000 | 100 |
| 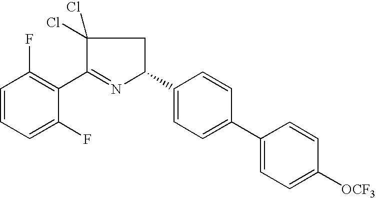 | 1000 | 100 |

### Example B

### Panonychus Test

| | |
|---|---|
| Solvent: | 3 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Plum trees (Prunus domestica) of a height of about 30 cm which are heavily infested by all stages of the fruit tree spider mite (Panonychus ulmi) are sprayed with a preparation of active compound of the desired concentration.

After the desired period of time, the effect in % is determined. 100% means that all spider mites have been killed; 0% means that none of the spider mites have been killed.

Active compounds, active compound concentrations and test results are shown in the table below.

TABLE B
Plant-damaging mites
Panonychus test

| | Active compound | |
|---|---|---|
| | concentration in | Kill rate in % |
| Active compounds | ppm | after 14^{d} |
| 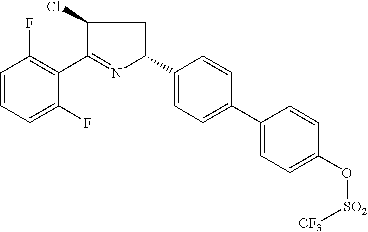 | 200 | 100 |

### Example C

### Phaedon larvae Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) whilst the leaves are still moist.

After the desired period of time, the kill in % is determnined. 100% means that all beetle larvae have been killed; 0% means that none of the beetle larvae have been killed.

Active compounds, active compound concentrations and test results are shown in the table below.

TABLE C
Plant-damaging insects
Phaedon larvae test

| | Active compound | |
|---|---|---|
| | concentration in | Kill rate in % |
| Active compounds | ppm | after 7^{d} |
| 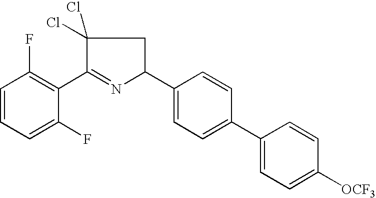 | 1000 | 100 |
| 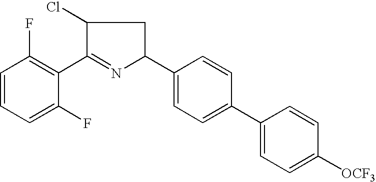 | 1000 | 100 |
| 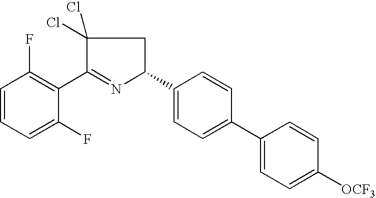 | 1000 | 100 |
| 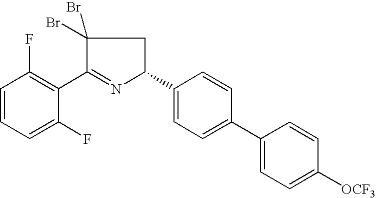 | 1000 | 100 |
| 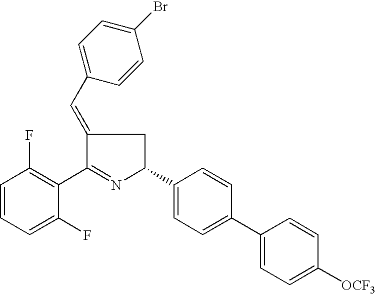 | 1000 | 100 |
| 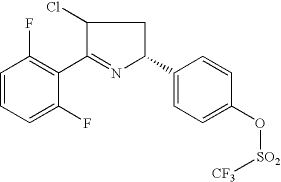 | 1000 | 100 |
| 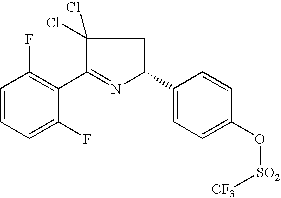 | 1000 | 100 |
| 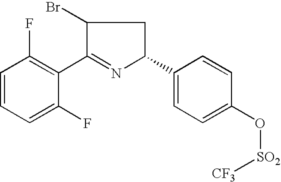 | 1000 | 100 |
| 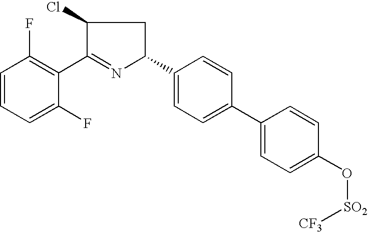 | 1000 | 100 |

### Example D

### Plutella Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamond-back moth (Plutella xylostella) whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE D
Plant-damaging insects
Plutella test

| | Active compound | |
|---|---|---|
| | concentration in | Kill rate in % |
| Active compounds | ppm | after 6^{d} |
| 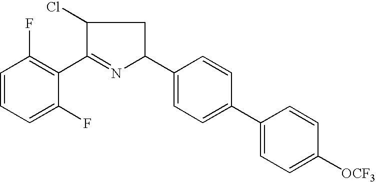 | 1000 | 100 |

### Example E

### Spodoptera exigua Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the army-worm (Spodoptera exigua) whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE E
Plant-damaging insects
Spodoptera exigua test

| | Active compound | |
|---|---|---|
| | concentration in | Kill rate in % |
| Active compounds | ppm | after 6^{d} |
| 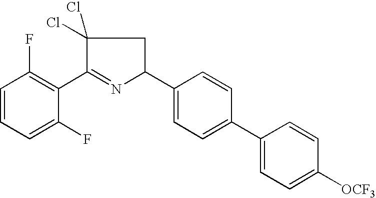 | 1000 | 100 |
| 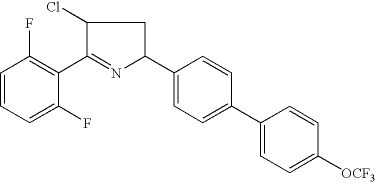 | 1000 | 100 |
| 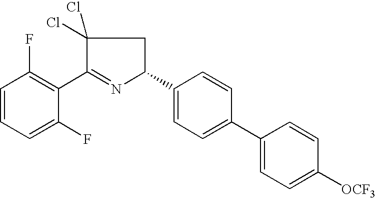 | 1000 | 100 |

### Example F

### Spodoptera frugiperda Test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the army-worm (Spodoptera frugiperda) whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE F
Plant-damaging insects
Spodoptera frugiperda test

| | Active compound | |
|---|---|---|
| | concentration in | Kill rate in % |
| Active compounds | ppm | after 7^{d} |
| 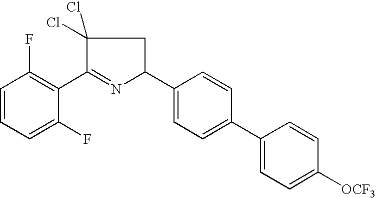 | 1000 | 100 |
| 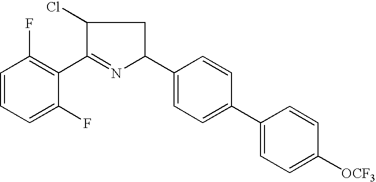 | 1000 | 100 |
| 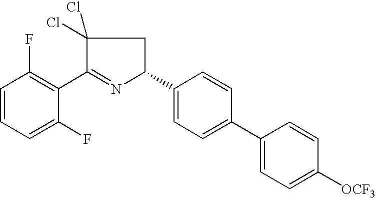 | 1000 | 100 |
| 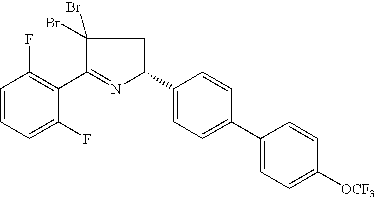 | 1000 | 100 |
| 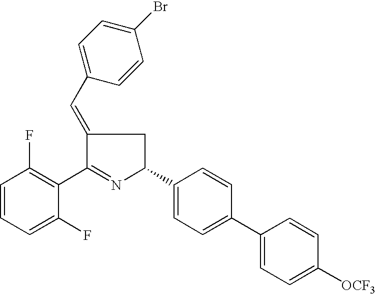 | 1000 | 100 |
| 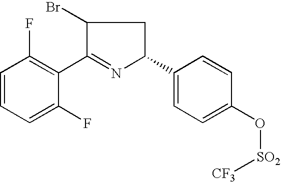 | 1000 | 100 |
| 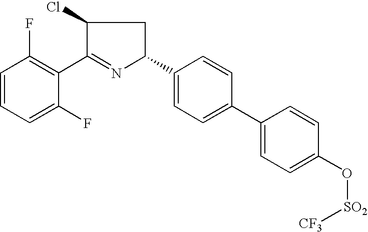 | 1000 | 100 |

### Example G

### Tetranychus Test (OP-Resistant/Dip Treatment)

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Bean plants (Phaseolus vulgaris) which are heavily infested by all stages of the greenhouse red spider mite (Tetranychus urticae) are dipped into a preparation of active compound of the desired concentration.

After the desired period of time, the effect in % is determined. 100% means that all spider mites have been killed; 0% means that none of the spider mites have been killed.

Active compounds, active compound concentrations and test results are shown in the table below.

TABLE G
Plant-damaging mites
Tetranychus test (OP-resistant/dip treatment)

| | Active compound | |
|---|---|---|
| | concentration in | Kill rate in % |
| Active compounds | ppm | after 7^{d} |
| 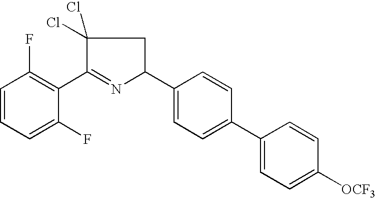 | 100 | 98 |
| 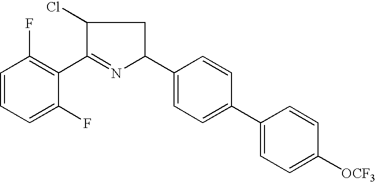 | 100 | 98 |
| 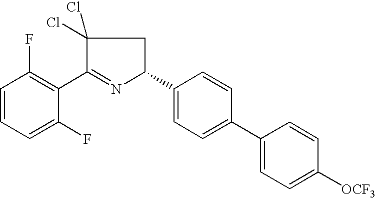 | 100 | 98 |
| 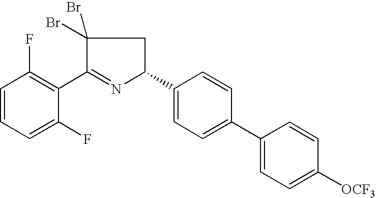 | 100 | 98 |
| 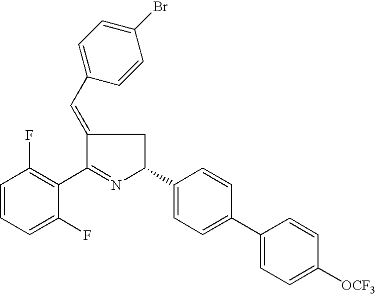 | 100 | 98 |
| 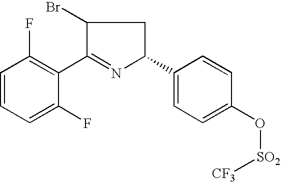 | 100 | 98 |
| 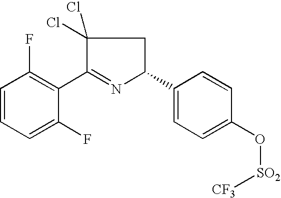 | 100 | 95 |
| 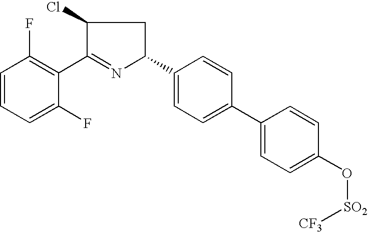 | 100 | 98 |

### Example H

### Blowfly Larvae Test/Development-Inhibitory Action

| | |
|---|---|
| Test animals: | Lucilia cuprina larvae |
| Solvent: | Dimethyl sulphoxide |

20 mg of active compound are dissolved in 1 ml of dimethyl sulphoxide, more dilute concentrations are prepared by dilution with dist. H₂O.

About 20 Lucilia cuprina larvae are introduced into a test tube which contains about 1 cm³ of horsemeat and 0.5 ml of the preparation of active compound to be tested. The activity of the preparation of active compound is determined after 24 and 48 hours. The test tubes are transferred into a beaker whose bottom is covered with sand. After a further 2 days, the test tubes are removed and the pupae are counted.

The activity of the preparation of active compound is assessed using the number of flies that have hatched after 1.5 times the development period of an untreated control. 100% means that none of the flies have hatched; 0% means that all of the flies have hatched normally.

Active compounds, active compound concentrations and test results are shown in the table below.

TABLE H
Blowfly larvae test
Development-inhibitory action

| | Active compound | |
|---|---|---|
| | concentration in | Kill rate in % |
| Active compounds | ppm | after 48 h |
| 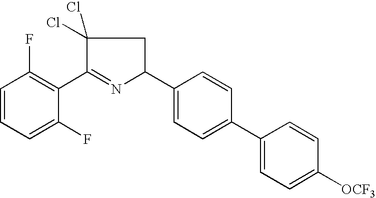 | 100 | 100 |
| 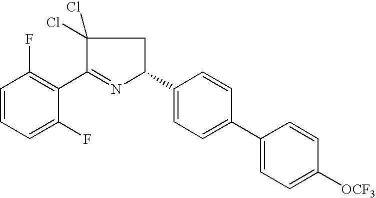 | 100 | 100 |
| 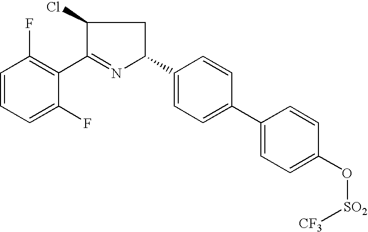 | 100 | 100 |

### Example J

### Nymph ecdysis Test on Polyphagous Ticks

| | |
|---|---|
| Test animals: | Amblyomma variegatum or A. hebraeum, nymphs |
| | which have sucked themselves full |
| Solvent: | Dimethyl sulphoxide |

20 mg of active compound are dissolved in 1 ml of dimethyl sulphoxide, more dilute concentrations are prepared by dilution with dist. H₂O.

10 nymphs which have sucked themselves full are immersed for 1 minute into the preparation of active compound to be tested. The animals are transferred to petri dishes (Ø0 9.5 cm) fitted with filter discs and covered. After the nymphs have remained in a controlled-environment cabinet for 4 to 6 weeks, the effect on ecdysis is determined.

100% means that none of the animals have undergone normal ecdysis. 0% means that all animals have undergone ecdysis.

Active compounds, active compound concentrations and test results are shown in the table below.

TABLE 3
Nymph ecdysis test
on polyphagous ticks

| | Active compound | |
|---|---|---|
| | concentration in | Effect on |
| Active compounds | ppm | ecdysis in % |
| 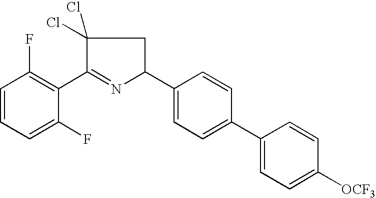 | 100 | 100 |
| 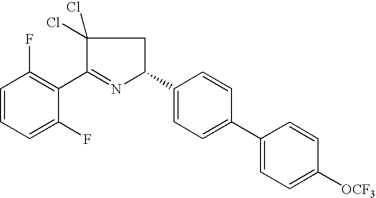 | 100 | 100 |
| 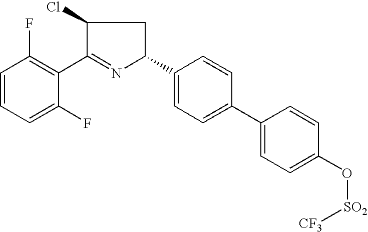 | 100 | 100 |

### Example K

### Diabrotica balteata Test (Larvae in the Soil)

Critical concentration test/soil insects—treatment of transgenic plants

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of the active compound in the preparation is virtually immaterial, only the amount by weight of active compound per volume unit of soil, which is stated in ppm (mg/l), matters. The soil is filled into 0.25 1 pots, and these are left standing at 20° C.

Immediately after the preparation, 5 pre-germinated maize corns of the cultivar YIELD GUARD (trade mark of Monsanto Comp., USA) are placed into each pot. After 2 days, the corresponding test insects are placed into the treated soil. After a further 7 days, the efficacy of the active compound is determined by counting the maize plants that have emerged (1 plant=20% activity).

### Example L

### Heliothis virescens Test (Treatment of Transgenic Plants)

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soyabean shoots (Glycine max) of the cultivar Roundup Ready (trade mark of Monsanto Comp. USA) are treated by being dipped into the preparation of active compound of the desired concentration and are populated with the tabacco bud worm Heliothis virescens whilst the leaves are still moist.

After the desired period of time, the kill in % is determined. 100% means that all caterpillars have been killed; 0% means that none of the caterpillars have been killed.

1. Δ¹-Pyrrolines of the formula (I) 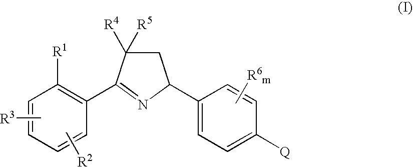
in which
R¹ represents halogen, represents in each case optionally substituted alkyl or alkoxy or represents —S(O)_{w}R⁷,
R² and R³ independently of one another represent hydrogen, halogen or represent in each case optionally substituted alkyl or alkoxy,
R⁴ represents halogen or optionally substituted alkyl or represents —SO₃R⁸,
R⁵ represents hydrogen, halogen or optionally substituted alkyl or represents —SO₃R⁸,
R⁴ and R⁵ furthermore together represent the grouping 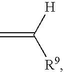
R⁶ represents halogen, represents in each case optionally substituted alkyl, alkoxy or alkylthio,
m represents 0, 1, 2, 3 or 4,
Q represents the grouping —X—Y-Z-E, with the proviso that Y does not represent a direct bond if X does not represent a direct bond,
R⁷ represents optionally substituted alkyl,
R⁸ represents hydrogen or optionally substituted alkyl,
R⁹ represents hydrogen or in each case optionally substituted alkyl, aryl or heteroaryl,
X represents a direct bond, oxygen, —S(O)_{w}—, —NR¹⁰—, carbonyl, carbonyloxy, oxycarbonyl, oxysulphonyl (OSO₂), alkylene, halogenoalkylene, alkenylene, halogenoalkenylene, alkinylene, alkylenoxy, oxyalkylene, oxyalkylenoxy, —S(O)_{w}-alkylene, cyclopropylene or oxiranylene,
Y represents a direct bond or represents in each case optionally substituted phenylene, naphthylene, tetrahydronaphthylene or heterocyclylene,
Z represents a direct bond or —(CH₂)ₙ—,
E represents hydrogen, halogen, hydroxyl, cyano, formyl, nitro, trialkylsilyl, pentafluorothio, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —C(R¹⁸)═N—OR¹⁹, —SO₂NR²⁰R²¹; or represents in each case optionally substituted alkyl, alkenyl, alkinyl, alkoxy, alkenyloxy, cycloalkyl, cycloalkylalkyl, cycloalkyloxy, aryl, arylalkyl, aryloxy, aryloxyalkyl, saturated or unsaturated heterocyclyl or heterocyclylalkyl,
R¹⁰ represents in each case optionally substituted alkyl, cycloalkyl, cycloalkylalkyl, aryl or arylalkyl,
R¹¹ represents in each case optionally substituted alkyl, cycloalkyl, aryl or arylalkyl,
R¹² and R¹³ independently of one another represent hydrogen or —SO₂R¹¹, represents in each case optionally substituted alkyl, alkenyl, cycloalkyl, cycloalkylalkyl, aryl, arylalkyl or saturated or unsaturated heterocyclyl or heterocyclylalkyl,
R¹² and R¹³ furthermore together represent in each case optionally substituted alkenylene or alkylene, where the alkylene chain may in each case be interrupted by —O—, —S— or —NR²²—,
R¹⁴ and R¹⁵ independently of one another represent hydrogen or —SO₂R¹¹, represent in each case optionally substituted alkyl, alkenyl, cycloalkyl, cycloalkylalkyl, aryl, arylalkyl or saturated or unsaturated heterocyclyl or heterocyclylalkyl,
R¹⁴ and R¹⁵ furthermore together represent optionally substituted alkylene, where the alkylene chain may be interrupted by —O—, —S— or —NR²²—,
R¹⁶ and R¹⁷ independently of one another represent hydrogen, represent in each case optionally substituted alkyl, cycloalkyl, cycloalkylalkyl, aryl or arylalkyl,
R¹⁶ and R¹⁷ furthermore together represent in each case optionally substituted alkylene or alkenylene,
R¹⁸ and R¹⁹ independently of one another represent hydrogen, represent in each case optionally substituted alkyl or alkenyl,
R²⁰and R²¹ independently of one another represent hydrogen, represent in each case optionally substituted alkyl or cycloalkyl,
R²⁰ and R²¹ furthermore together represent in each case optionally substituted alkylene, alkoxyalkylene or alkylthioalkylene,
R²² represents hydrogen, —SO₂R¹¹, —COR¹¹or —CO₂R¹¹; represents in each case optionally substituted alkyl, alkenyl, cycloalkyl, aryl, or saturated or unsaturated heterocyclyl,
w represents 0, 1 or 2,
n represents 1, 2, 3 or 4.

2. Δ¹-Pyrrolines of the formula (I) according to claim 1, in which
R¹ represents halogen, alkyl, halogenoalkyl, alkoxy, halogenoalkoxy or —S(O)_{w}R⁷,
R² and R³ independently of one another represent hydrogen, halogen, alkyl, halogenoalkyl, alkoxy, halogenoalkoxy or alkoxyalkyl,
R⁴ represents halogen, alkyl halogenoalkyl or represents
R⁵ represents hydrogen, halogen, alkyl, halogenoalkyl or represents —SO₃R⁸,
R⁴ and R⁵ furthermore together represent the grouping 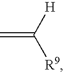
R⁶ represents halogen, alkyl, alkoxy, alkylthio, halogenoalkyl, halogenoalkoxy or halogenoalkylthio,
m represents 0, 1, 2, 3 or 4,
Q represents the grouping —X—Y-Z-E, with the proviso that Y does not represent a direct bond if X does not represent a direct bond,
R⁷ represents alkyl or halogenoalkyl,
R⁸ represents hydrogen, alkyl or halogenoalkyl,
R⁹ represents hydrogen, alkyl, halogenoalkyl or represents aryl or 5- to 10-membered heteroaryl having one or more heteroatoms from the group consisting of nitrogen, oxygen and sulphur, each of which radicals is optionally mono- to tetrasubstituted by identical or different substituents from the list W¹,
X represents a direct bond, oxygen, —S(O)_{w}—, —NR¹⁰—, carbonyl, carbonyloxy, oxycarbonyl, oxysulphonyl (OSO₂), alkylene, halogenoalkylene, alkenylene, halogenoalkenylene, alkinylene, alkylenoxy, oxyalkylene, oxyalkylenoxy, —S(O)_{w}-alkylene, cyclopropylene or oxiranylene,
Y represents a direct bond or represents phenylene, naphthylene, tetrahydronaphthylene or 5- to 10-membered saturated or unsaturated heterocyclylene having one or more heteroatoms from the group consisting of nitrogen, oxygen and sulphur, each of which radicals is optionally mono- to tetrasubstituted by identical or different radicals from the list W¹,
Z represents a direct bond or —(CH₂)ₙ—,
E represents hydrogen, halogen, hydroxyl, cyano, formyl, nitro, trialkylsilyl, pentafluorothio, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —C(R¹⁸)═N—OR¹⁹, —SO₂NR²⁰R²¹; represents alkyl, alkenyl, alkinyl, alkoxy, alkenyloxy, each of which is optionally mono- or polysubstituted by identical or different substituents from the group consisting of halogen, cyano, alkoxy and —NR¹²R¹³; or represents cycloalkyl, cycloalkylalkyl, cycloalkyloxy, aryl, arylalkyl, aryloxy, aryloxyalkyl, saturated or unsaturated 5- to 10-membered heterocyclyl or heterocyclylalkyl having one or more heteroatoms from the group consisting of nitrogen, oxygen and sulphur, each of which radicals is mono- or polysubstituted by identical or different substituents from the group consisting of halogen, cyano, nitro, alkyl, halogenoalkyl, alkenyl, halogenoalkenyl, alkoxy, halogenoalkoxy, alkylthio and halogenoalkylthio,
W¹ represents halogen, cyano, formyl, nitro, trialkylsilyl, alkyl, halogenoalkyl, alkoxy, halogenoalkoxy, alkenyl, halogenoalkenyl, alkenyloxy, halogenoalkenyloxy, alkylcarbonyl, alkoxycarbonyl, —S(O)_{w}R¹¹, —C(R¹⁸)═N—OR¹⁹, —SO₂NR²⁰R²¹, —(CH₂)ₚNR²⁰R²¹, —(CH₂)ₚN(R²⁰)COR²¹, —(CH₂)ₚN(R²⁰)SO₂R²¹, —OSO₂R²⁰, —OSO₂NR²⁰R²¹,
R¹⁰ represents alkyl, halogenoalkyl, or represents cycloalkyl, cycloalkylalkyl, aryl or arylalkyl, each of which is optionally mono- or polysubstituted by identical or different substituents from the group consisting of halogen, alkyl, halogenoalkyl, alkoxy, halogenoalkoxy, alkylthio and halogenoalkylthio,
R¹¹ represents alkyl which is optionally mono- or polysubstituted by identical or different substituents from the group consisting of halogen and —NR¹²R¹³, represents cycloalkyl, aryl or arylalkyl, each of which is optionally mono- or polysubstituted by identical or different substituents from the group consisting of halogen, cyano, alkyl, halogenoalkyl, alkoxy, halogenoalkoxy, alkylthio and halogenoalkylthio,
R¹² and R¹³ independently of one another represent hydrogen or —SO₂R¹¹, represent alkyl or alkenyl, each of which is optionally mono- or polysubstituted by identical or different substituents from the group consisting of halogen, alkylcarbonyl, alkylcarbonyloxy, alkylamino, dialkylamino, alkoxy, halogenoalkoxy, alkylthio and halogenoalkylthio; represents cycloalkyl, cycloalkylalkyl, aryl, arylalkyl, saturated or unsaturated 5- to 10-membered heterocyclyl or heterocyclylalkyl having one or more heteroatoms from the group consisting of nitrogen, oxygen and sulphur, each of which radicals is optionally mono- or polysubstituted by identical or different substituents from the group consisting of halogen, cyano, alkyl, halogenoalkyl, alkoxy, halogenoalkoxy, alkylthio and halogenoalkylthio,
R¹² and R¹³ furthermore together represent alkenylene which is optionally mono- or polysubstituted by identical or different substituents from the group consisting of halogen, cyano, alkoxy, halogenoalkoxy, alkylthio and halogenoalkylthio or represent alkylene, where the alkylene chain may be interrupted by —O—, —S— or —NR²²—, and where the radical is optionally mono- or polysubstituted by identical or different substituents from the group consisting of halogen, cyano, alkyl, halogenoalkyl, alkoxy, halogenoalkoxy, alkylthio and halogenoalkylthio,
R¹⁴ and R¹⁵ independently of one another represent hydrogen, —SO₂R¹¹, represent alkyl or alkenyl, each of which is optionally mono- or polysubstituted by identical or different substituents from the group consisting of halogen, alkylamino, dialkylamino, alkoxy and alkylthio; represents cycloalkyl, cycloalkylalkyl, aryl, arylalkyl, saturated or unsaturated 5- to 10-membered heterocyclyl or heterocyclylalkyl having one or more heteroatoms from the group consisting of nitrogen, oxygen and sulphur, each of which radicals is optionally mono- or polysubstituted by identical or different substituents from the group consisting of halogen, cyano, alkyl, halogenoalkyl, alkoxy, halogenoalkoxy, alkylthio and halogenoalkylthio,
R¹⁴ and R¹⁵ furthermore together represent alkylene, where the alkylene chain may be interrupted by —O—, —S— or —NR²²—, and where the radical is optionally mono- or polysubstituted by identical or different substituents from the group consisting of halogen, cyano, alkyl, halogenoalkyl, alkoxy, halogenoalkoxy, alkylthio and halogenoalkylthio,
R¹⁶ and R¹⁷ independently of one another represent hydrogen, represent alkyl which is optionally mono- or polysubstituted by identical or different substituents from the group consisting of halogen, cyano, alkoxy and alkylthio, represent cycloalkyl, cycloalkylalkyl, aryl or arylalkyl, each of which is optionally mono- or polysubstituted by identical or different substituents from the group consisting of halogen, cyano, alkyl, halogenoalkyl, alkoxy, halogenoalkoxy, alkylthio and halogenoalkylthio,
R¹⁶ and R¹⁷ furthermore together represent alkylene or alkenylene, each of which is optionally mono- or polysubstituted by identical or different substituents from the group consisting of halogen, alkyl, halogenoalkyl, alkoxy, halogenoalkoxy, alkylthio and halogenoalkylthio,
R¹⁸ and R¹⁹ independently of one another represent hydrogen, alkyl, alkenyl, halogenoalkyl or halogenoalkenyl,
R²⁰and R²¹ independently of one another represent hydrogen, alkyl, halogenoalkyl or represent cycloalkyl which is optionally mono- or polysubstituted by identical or different substituents from the group consisting of halogen and alkyl,
R²⁰ and R²¹ furthermore together represent alkylene, alkoxyalkylene or alkylthioalkylene, each of which is optionally mono- or polysubstituted by identical or different substituents from the group consisting of halogen and alkyl,
R²² represents hydrogen, —SO₂R¹¹, —COR¹¹ or —CO₂R¹¹; represents alkyl or alkenyl, each of which is optionally mono- or polysubstituted by identical or different substituents from the group consisting of halogen, cyano, alkylamino, dialkylamino, alkoxy, halogenoalkoxy, alkylthio and halogenoalkylthio; represents cycloalkyl, cycloalkylalkyl, aryl, arylalkyl, saturated or unsaturated 5- to 10-membered heterocyclyl or heterocyclylalkyl having one or more heteroatoms from the group consisting of nitrogen, oxygen and sulphur, each of which radicals is optionally mono- or polysubstituted by identical or different substituents from the group consisting of halogen, cyano, alkyl, halogenoalkyl, alkoxy, halogenoalkoxy, alkylthio and halogenoalkylthio,
w represents 0, 1 or 2,
n represents 1, 2, 3 or 4,
p represents 0, 1 or 2.

3. Δ¹-Pyrrolines of the formula (I) according to claim 1, in which
R¹ represents halogen, C₁-C₆-alkyl, C₁-C₆-halogenoalkyl, C₁-C₆-alkoxy, C₁-C₆-halogenoalkoxy or —S(O)_{w}R⁷,
R² and R³ independently of one another represent hydrogen, halogen, C₁-C₆-alkyl, C₁-C₆-halogenoalkyl, C₁-C₆-alkoxy, C₁-C₆-halogenoalkoxy or C₁-C₆-alkoxy-C₁-C₆-alkyl,
R⁴ represents halogen, C₁-C₁₀-alkyl, C₁-C₁₀-halogenoalkyl or represents —SO₃R⁸,
R⁴ and R⁵ furthermore together represent the grouping 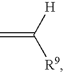
R⁵ represents hydrogen, halogen, C₁-C₁₀-alkyl, C₁-C₁₀-halogenoalkyl or represents —SO₃R⁸,
R⁶ represents fluorine, chlorine, bromine, C₁-C₆-alkyl, C₁-C₆-alkoxy, C₁-C₆-alkylthio, C₁-C₆-halogenoalkyl, C₁-C₆-halogenoalkoxy or C₁-C₆-halogenoalkylthio,
m represents 0, 1, 2 or3,
Q represents the grouping —X—Y-Z-E, with the proviso that Y does not represent a direct bond if X does not represent a direct bond,
R⁷ represents C₁-C₆-alkyl or C₁-C₆-halogenoalkyl,
R⁸ represents hydrogen, C₁-C₆-alkyl or C₁-C₆-halogenoalkyl,
R⁹ represents hydrogen, C₁-C₆-alkyl, C₁-C₆-halogenoalkyl, or represents aryl or 5- or 6-membered heteroaryl having 1 to 3 heteroatoms, which contains 0 to 3 nitrogen atoms, 0 to 2 non-adjacent oxygen atoms and
0 to 2 non-adjacent sulphur atoms (in particular furyl, thienyl, pyrrolyl, oxazolyl, thiazolyl or pyridinyl), each of which radicals is optionally mono- to tetrasubstituted by identical or different radicals from the list W¹,
X represents a direct bond, oxygen, —S(O)_{w}—, —NR¹⁰—, carbonyl, carbonyloxy, oxycarbonyl, oxysulphonyl (OSO₂), C₁-C₆-alkylene, C₁-C₆-halogenoalkylene, C₂-C₆-alkenylene, C₂-C₆-halogenoalkenylene, C₂-C₆-alkinylene, C₁-C₆-alkylenoxy, C₁-C₆-oxyalkylene, oxy-C₁-C₆-alkylenoxy or —S(O)_{w}-C₁-C₆-alkylene,
Y represents a direct bond or represents 1,4-phenylene, 1,3-phenylene, 1,2-phenylene, 2,6-naphthylene, 2,7-naphthylene, 1,4-naphthylene, 2,6-(1,2,3,4-tetrahydro)naphthylene, 2,7-(1,2,3,4-tetrahydro)-naphthylene, 1,4-(1,2,3,4-tetrahydro)naphthylene, 5,8-(1,2,3,4-tetrahydro)naphthylene, each of which is optionally mono- to tetrasubstituted by identical or different radicals from the list W¹; or represents 5- or 6-membered saturated or unsaturated heterocyclylene having 1 to 3 heteroatoms, which contains 0 to 3 nitrogen atoms, 0 to 2 non-adjacent oxygen atoms and/or 0 to 2 non-adjacent sulphur atoms (in particular furylene, thienylene, pyrrolylene, oxazolylene, thiazolylene, pyridinylene, pyrimidinylene or pyridazinylene or pyrazinylene), each of which radicals is optionally mono- to tetrasubstituted by identical or different radicals from the list W¹,
Z represents a direct bond or —(CH₂)ₙ—,
E represents hydrogen, fluorine, chlorine, bromine, hydroxyl, cyano, formyl, nitro, tri-(C₁-C₆-alkyl)silyl, pentafluorothio, —S(O)_{w}R¹¹, —OSO₂R¹¹, —NR¹²R¹³, —COR¹¹, —CO₂R¹¹, —OC(O)R¹¹, —CONR¹⁴R¹⁵ , —N(R¹⁶)COR¹⁷, —C(R¹⁸)═N—OR¹⁹, —SO₂NR²⁰R²¹; represents C₁-C₂₀alkyl, C₂-C₂₀-alkenyl, C₂-C₁₀-alkinyl, C₁-C₂₀-alkoxy, C₂-C₂₀-alkenyloxy, each of which is optionally mono- or polysubstituted by identical or different substituents from the group consisting of halogen, cyano, C₁-C₁₀-alkoxy and —NR¹²R¹³; or represents C₃-C₁₂-cycloalkyl, C₃-C₇-cycloalkyl-C₁-C₄-alkyl, C₃-C₁₂-cycloalkyloxy, aryl, aryl-C₁-C₄-alkyl, aryloxy, aryloxy-C₁-C₄-alkyl, saturated or unsaturated 5- to 10-membered heterocyclyl or heterocyclyl-C₁-C₄alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidino, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, piperidino, morpholinyl, thiomorpholinyl, morpholino, thiomorpholino, triazinyl, triazolyl, quinolinyl or isoquinolinyl), each of which radicals is optionally mono- to tetrasubstituted by identical or different radicals from the group consisting of halogen, cyano, nitro, C₁-C₆-alkyl, C₁-C₆-halogenoalkyl, C₂-C₆-alkenyl, C₂-C₆-halogenoalkenyl, C₁-C₆-alkoxy, C₁-C₆-halogenoalkoxy, C₁-C₆-alkylthio and C₁-C₆-halogenoalkylthio,
W¹ represents fluorine, chlorine, bromine, cyano, formyl, nitro, tri-(C₁-C₆alkyl)silyl, C₁-C₆-alkyl, C₁-C₆-halogenoalkyl, C₁-C₆-alkoxy, C₁-C₆-halogenoalkoxy, C₂-C₆-alkenyl, C₂-C₆-halogenoalkenyl, C₂-C₆-alkenyloxy, C₂-C₆-halogenoalkenyloxy, C₁-C₆-alkylcarbonyl, C₁-C₆-alkoxycarbonyl, —S(O)_{w}R¹¹, —C(R¹⁸)═N—OR¹⁹, —SO₂NR²⁰R²¹, —(CH₂)ₚNR²⁰R²¹, —(CH₂)ₚN(R²⁰)COR^{2 1}, —(CH₂)ₚN(R²⁰)SO₂R²¹, —OSO₂R²⁰, —OSO₂NR²⁰R²,
R¹⁰ represents C₁-C₆-alkyl, C₁-C₆-halogenoalkyl, or represents C₃-C₇-cycloalkyl, C₃-C₆-cycloalkyl-C₁-C₄-alkyl, aryl or aryl-C₁-C₄-alkyl, each of which is mono- to tetrasubstituted by identical or different substituents from the group consisting of halogen, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio,
R¹¹ represents C₁-C₂₀-alkyl, each of which is optionally mono- or polysubstituted by identical or different substituents from the group consisting of halogen and —NR¹²R¹³, represents C₃-C₆-cycloalkyl, aryl or aryl-C₁-C₄-alkyl, each of which is optionally mono- to octasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio,
R¹² and R¹³ independently of one another represent hydrogen, —SO₂R¹¹, represent C₁-C₂₀-alkyl or C₂-C₂₀-alkenyl, each of which is optionally mono- or polysubstituted by identical or different substituents from the group consisting of halogen, C₁-C₆-alkylcarbonyl, C₁-C₆-alkylcarbonyloxy, C₁-C₆-alkylamino, di-(C₁-C₆-alkyl)amino, C₁-C₆-alkoxy, C₁-C₆-halogenoalkoxy, C₁-C₆-alkylthio and C₁-C₆-halogenoalkylthio; represent C₃-C₁₂-cycloalkyl, C₃-C₇-cycloalkyl-C₁-C₄-alkyl, aryl, aryl-C₁-C₄-alkyl, saturated or unsaturated 5- to 10-membered heterocyclyl or heterocyclyl-C₁-C₄-alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl), each of which radicals is optionally mono- to tetrasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio,
R¹² and R¹³ furthermore together represent C₂-C₁₂-alkenylene which is optionally mono- or polysubstituted by identical or different substituents from the group consisting of halogen, cyano, C₁-C₆-alkoxy, C₁-C₆-halogenoalkoxy, C₁-C₆-alkylthio and C₁-C₆-halogenoalkylthio or represent C₃-C₁₂-alkylene, where the alkylene chain may be interrupted by —O—, —S— or —NR²²—, and where the radical is optionally mono- or polysubstituted in the alkylene moiety by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio,
R¹⁴ and R¹⁵ independently of one another represent hydrogen, —SO₂R¹¹, represent C₁-C₆-alkyl or C₂-C₆-alkenyl, each of which is optionally mono- to tridecasubstituted by identical or different substituents from the group consisting of halogen, C₁-C₆-alkylamino, di-(C₁-C₆-alkyl)amino, C₁-C₆-alkoxy, C₁-C₆-halogenoalkoxy, C₁-C₆-alkylthio and C₁-C₆-halogenoalkylthio; represent C₃-C₇-cycloalkyl, C₃-C₇-cycloalkyl-C₁-C₄-alkyl, aryl, aryl-C₁-C₄-alkyl, saturated or-unsaturated 5- to 10-membered heterocyclyl or heterocyclyl-C₁-C₄-alkyl having 1 to 4 heteroatoms, which contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl), each of which is optionally mono- to tetrasubstituted by identical or different substituents from the group consisting of halogen, cyano, C₁-C₆-alkyl, C₁-C₆-halogenoalkyl, C₁-C₆-alkoxy, C₁-C₆-halogenoalkoxy C₁-C₆-alkylthio and C₁-C₆-halogenoalkylthio,
R¹⁴ and R¹⁵ furthermore together represent C₃-C₆-alkylene which is optionally mono- to tetrasubstituted in the alkylene moiety by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio, represent —(CH₂)₂—O—(CH₂)₂—, —(CH₂)₂—S—(CH₂)₂— or —(CH₂)₂—N(R²²)—(CH₂,
R¹⁶ and R¹⁷ independently of one another represent hydrogen, represent C₁-C₆-alkyl which is optionally mono- to tridecasubstituted by identical or different substituents from the group consisting of halogen, cyano, C₁-C₆-alkoxy and C₁-C₆-alkylthio, represent C₃-C₇-cycloalkyl, C₃-C₇-cycloalkyl-C₁-C₄-alkyl, aryl or aryl-C₁-C₄-alkyl, each of which is optionally mono- to octasubstituted by identical or different substituents from the group consisting of halogen, cyano, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio,
R¹⁶ and R¹⁷ furthermore together represent C₃-C₁₀-alkylene or C₃-C₁₀-alkenylene, each of which is optionally mono- to octasubstituted by identical or different substituents from the group consisting of halogen, C₁-C₆-alkyl, C₁-C₆-halogenoalkyl, C₁-C₆-alkoxy, C₁-C₆-halogenoalkoxy, C₁-C₆-alkylthio and C₁-C₆-halogenoalkylthio,
R¹⁸ and R¹⁹ independently of one another represent hydrogen, C₁-C₆-alkyl, C₂-C₆-alkenyl, C₁-C₆-halogenoalkyl or C₂-C₆-halogenoalkenyl,
R²⁰ and R²¹ independently of one another represent hydrogen, C₁-C₆-alkyl, C₁-C₆-halogenoalkyl or represent C₃-C₇-cycloalkyl which is optionally mono- to octasubstituted by identical or different substituents from the group consisting of fluorine, chlorine, bromine and C₁-C₆-alkyl,
R²⁰ and R²¹ furthermore together represent C₃-C₆-alkylene, C₁-C₃-alkoxy-C₁-C₃-alkylene or C₁-C₃-alkylthio-C₁-C₃-alkylene, each of which is optionally mono- to nonasubstituted by identical or different substituents from the group consisting of fluorine, chlorine, bromine and C₁-C₆-alkyl,
R²² represents hydrogen, —SO₂R¹¹, —COR¹¹ or —CO₂R¹¹; represents C₁-C₂₀-alkyl or C₂-C₂₀-alkenyl, each of which is optionally mono- or polysubstituted by identical or different substituents from the group consisting of halogen, cyano, C₁-C₆-alkylamino, di-(C₁-C₆-alkyl)amino, C₁-C₆-alkoxy, C₁-C₆-halogenoalkoxy, C₁-C₆-alkylthio and C₁-C₆-halogenoalkylthio; represents C₃-C₁₂-cycloalkyl, C₃-C₇-cycloalkyl-C₁-C₄-alkyl, aryl, aryl-C₁-C₄-alkyl, saturated or unsaturated 5- to 10-membered heterocyclyl or heterocyclyl-C₁-C₄-alkyl having 1 to 4 heteroatoms, which radicals contain 0 to 4 nitrogen atoms, 0 to 2 non-adjacent oxygen atoms and/or 0 to 2 non-adjacent sulphur atoms (in particular tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl), each of which radicals is optionally mono- to tetrasubstituted by identical or different radicals from the group consisting of halogen, cyano, C₁-C₆-alkyl, C₁-C₆halogenoalkyl, C₁-C₆-alkoxy, C₁-C₆-halogenoalkoxy, C₁-C₆-alkylthio and C₁-C₆-halogenoalkylthio,
w represents 0, 1 or 2,
n represents 1, 2 or 3,
p represents 0, 1 or 2.

4. Δ¹-Pyrrolines of the formula (I) according to claim 1, in which
R¹ represents fluorine, chlorine, bromine, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms or —S(O)_{w}R⁷,
R² and R³ independently of one another represent hydrogen, fluorine, chlorine, bromine, C₁-C₄-alkyl, C₁-C₄-halogenoalkyl having 1 to 9 fluorine, chlorine and/or bromine atoms, C₁-C₄-alkoxy or C₁-C₄-halogenoalkoxy having 1 to 9 fluorine, chlorine and/or bromine atoms,
R⁴ represents fluorine, chlorine, bromine, C₁-C₆-alkyl, C₁-C₆-halogenoalkyl having 1 to 13 fluorine, chlorine and/or bromine atoms, or represents —SO₃R⁸,
R⁵ represents hydrogen, fluorine, chlorine, bromine, C₁-C₆-alkyl, C₁-C₆-halogenoalkyl having 1 to 13 fluorine, chlorine and/or bromine atoms, or represents —SO₃R⁸,
R⁴ and R⁵ furthermore together represent the grouping 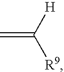
R⁶ represents fluorine, chlorine, C₁-C₄-alkyl, C₁-C₄-alkoxy, C₁-C₄-alkylthio; C₁-C₄-halogenoalkyl, C₁-C₄-halogenoalkoxy, C₁-C₄-halogenoalkylthio having in each case 1 to 9 fluorine, chlorine and/or bromine atoms,
m represents 0, 1 or 2,
Q represents the grouping —X—Y-Z-E, with the proviso that Y does not represent a direct bond if X does not represent a direct bond,
R⁷ represents C₁-C₄-alkyl or represents in each case fluorine- or chlorine-substituted methyl or ethyl,
R⁸ represents hydrogen, C₁-C₄-alkyl or C₁-C₄-halogenoalkyl having 1 to 9 fluorine, chlorine and/or bromine atoms,
R⁹ represents hydrogen, C₁-C₄-alkyl, C₁-C₄-halogenoalkyl having 1 to 9 fluorine, chlorine and/or bromine atoms, or represents phenyl, furyl, thienyl, pyrrolyl, oxazolyl, thiazolyl or pyridinyl, each of which is optionally mono- to trisubstituted by radicals from the list W¹,
X represents a direct bond, oxygen, —S(O)_{w}—, —NR¹⁰—, carbonyl, carbonyloxy, oxycarbonyl, oxysulphonyl (OSO₂), C₁-C₄-alkylene, C₁-C₄-halogenoalkylene having 1 to 8 fluorine, chlorine and/or bromine atoms, C₂-C₄-alkenylene, C₂-C₄-halogenoalkenylene having 1 to 6 fluorine, chlorine and/or bromine atoms, C₂-C₄-alkinylene, C₁-C₄-alkylenoxy, oxy-C₁-C₄-alkylene, oxy-C₁-C₄-alkylenoxy or —S(O)_{w}—C₁-C₄-alkylene,
Y represents a direct bond or represents 1,4-phenylene, 1,3-phenylene, 1,2-phenylene, 2,6-naphthylene, 2,7-naphthylene, 1,4-naphthylene, 2,6-(1,2,3,4-tetrahydro)naphthylene, 2,7-(1,2,3,4-tetrahydro)-naphthylene, 1,4-(1,2,3,4-tetrahydro)naphthylene, 5,8-(1,2,3,4-tetrahydro)naphthylene, 2,4-furylene, 2,4-thienylene, 2,4-pyrrolylene, 2,5-oxazolylene, 2,5-thiazolylene, 2,5-pyridinylene, 2,6-pyridinylene, 2,5-pyrimidinylene or 3,6-pyridazinylene or 2,5-pyrazinylene, each of which is optionally mono- to trisubstituted by identical or different radicals from the list W¹,
Z represents a direct bond or —(CH₂)ₙ—,
E represents hydrogen, fluorine, chlorine, bromine, hydroxyl, cyano, formyl, nitro, trimethylsilyl, dimethyl-tert-butylsilyl, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹³, —SO₂NR²⁰R²¹; represents C₁-C₁₆-alkyl, C₂-C₁₆-alkenyl, C₂-C₆-alkinyl, C₁-C₁₆-alkoxy, C₂-C₁₆-alkenyloxy, each of which is optionally mono- or polysubstituted by identical or different radicals from the group consisting of fluorine, chlorine, bromine, cyano, C₁-C₆-alkoxy and —NR¹²R¹³; or represents C₃-C₁₀-cycloalkyl, C₃-C₆-cycloalkyl-C_{1-C}₄-alkyl, C₃-C₁₀-cycloalkyloxy, phenyl, phenoxy, benzyl, phenylethyl, benzyloxy, tetrazolyl, furyl, furfuryl, benzofuryl, tetrahydrofuryl, thienyl, thenyl, benzothienyl, thiolanyl, pyrrolyl, indolyl, pyrrolinyl, pyrrolidino, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, piperidino, morpholinyl, thiomorpholinyl, morpholino, thiomorpholino, triazinyl, triazolyl, quinolinyl or isoquinolinyl, each of which is optionally mono- to trisubstituted by identical or different radicals from the group consisting of fluorine, chlorine, bromine, cyano, C₁-C₄-alkyl, C₁-C₄-halogenoalkyl having 1 to 9 fluorine, chlorine and/or bromine atoms, C₂-C₆-alkenyl, C₂-C₆-halogenoalkenyl having 1 to 8 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
W¹ represents fluorine, chlorine, bromine, cyano, formyl, trimethylsilyl, dimethyl-tert-butylsilyl, C₁-C₄-alkyl, C₁-C₄-alkoxy, C₂-C₄-alkenyl, C₂-C₄-alkenyloxy; represents C₁-C₄-halogenoalkyl, C₁-C₄-halogenoalkoxy having in each case 1 to 9 fluorine, chlorine and/or bromine atoms, C₂-C₄-halogenoalkenyl, C₂-C₄-halogenoalkenyloxy having in each case 1 to 8 fluorine, chlorine and/or bromine atoms; represents C₁-C₄-alkylcarbonyl, C₁-C₄-alkoxycarbonyl, —S(O)_{w}R¹¹, —SO₂NR²⁰R²¹, —(CH₂)ₚNR²⁰R²¹, —(CH₂)ₚN(R²⁰)COR²¹, —(CH₂)ₚN(R²⁰)SO₂R²¹, —OSO₂R²⁰, —OSO₂NR²⁰R²¹,
R¹⁰ represents methyl, ethyl, n-propyl, isopropyl, n-butyl, isobutyl, sec-butyl, tert-butyl, trifluoromethyl, trifluoroethyl, or represents cyclopropyl, cyclopropylmethyl, cyclopentyl, cyclopentylmethyl, cyclohexyl, cyclohexylmethyl, phenyl, benzyl or phenylethyl, each of which is optionally mono- to tetrasubstituted by identical or different substituents from the group consisting of fluorine, chlorine, bromine, C₁-C₄-alkyl, C₁-C₄-alkoxy, C₁-C₄-alkylthio and C₁-C₄-halogenoalkyl, C₁-C₄-halogenoalkoxy and C₁-C₄-halogenoalkylthio having in each case 1 to 9 fluorine, chlorine and/or bromine atoms,
R¹¹ represents C₁-C₁₀-alkyl which is optionally mono- or polysubstituted by identical or different substituents from the group consisting of fluorine, chlorine, bromine and —NR¹²R¹³, represents cyclopropyl, cyclopentyl, cyclohexyl, phenyl or benzyl, each of which is optionally mono- to tetrasubstituted by identical or different substituents from the group consisting of fluorine, chlorine, bromine, cyano, C₁-C₄-alkyl, C₁-C₄-alkoxy, C₁-C₄-alkylthio and C₁-C₄-halogenoalkyl, C₁-C₄-halogenoalkoxy and C₁-C₄-halogenoalkylthio having in each case 1 to 9 fluorine, chlorine and/or bromine atoms,
R¹² and R¹³ independently of one another represent hydrogen, —SO₂R¹¹, represent C₁-C₁₆-alkyl or C₂-C₁₆-alkenyl, each of which is optionally mono- or polysubstituted by identical or different substituents from the group consisting of fluorine, chlorine, bromine, C₁-C₄-alkylcarbonyl, C₁-C₄-alkylcarbonyloxy, C₁-C₄-alkylamino, di-(C₁-C₄-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₁₀-cycloalkyl, C₃-C₆-cycloalkyl-C₁-C₄-alkyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R¹² and R¹³ :furthermore together represent C₂-C₁₀-alkenylene, which is optionally mono- or polysubstituted by identical or different substituents from the group consisting of fluorine, chlorine, bromine, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms or represents C₃-C₁₀-alkylene, where the alkylene chain may be interrupted by —O—, —S— or —NR²²—, and where the radicals are optionally mono- or polysubstituted in the alkylene moiety by identical or different substituents from the group consisting of fluorine, chlorine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R¹⁴ and R¹⁵ independently of one another represent hydrogen, —SO₂R¹¹, represent C₁-C₆-alkyl or C₂-C₆-alkenyl, each of which is optionally mono- to nonasubstituted by identical or different substituents from the group consisting of fluorine, chlorine, bromine, C₁-C₄-alkylamino, di-(C₁-C₄-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₆-cycloalkyl, C₃-C₆-cycloalkyl-C₁-C₄-alkyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, C₁-C₄-alkyl, cyano, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R¹⁴ and R¹⁵ furthermore together represent C₄-C₅-alkylene, —(CH₂)₂—O—(CH₂)₂—, —(CH₂)₂—S—(CH₂)₂—, —(CH₂)₂—N(R²²)—(CH₂)₂— which is in each case optionally mono- to tetrasubstituted in the alkylene moiety by identical or different substituents from the group consisting of fluorine, chlorine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R¹⁶ and R¹⁷ independently of one another represent hydrogen, represent C₁-C₆-alkyl which is optionally mono- to nonasubstituted by identical or different substituents from the group consisting of fluorine, chlorine, bromine, C₁-C₄-alkoxy and C₁-C₄-alkylthio, represent C₃-C₆-cycloalkyl, C₃-C₆-cycloalkyl-C₁-C₄-alkyl, phenyl, benzyl or phenylethyl, each of which is optionally mono- to tetrasubstituted by identical or different substituents from the group consisting of fluorine, chlorine, bromine, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R¹⁶ and R¹⁷ furthermore together represent C₃-C₈-alkylene or C₃-C₈alkenylene, each of which is optionally mono- to tetrasubstituted by identical or different substituents from the group consisting of fluorine, chlorine, bromine, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R²⁰ and R²¹ independently of one another represent hydrogen, C₁-C₄-alkyl, C₁-C₄-halogenoalkyl having 1 to 9 fluorine, chlorine and/or bromine atoms or represent C₃-C₆-cycloalkyl which is optionally mono- to tetrasubstituted by identical or different substituents from the group consisting of fluorine, chlorine, bromine and C₁-C₄-alkyl,
R^{20 and R}²¹ furthermore together represent —(CH₂)₃—, —(CH₂)₄—, —(CH₂)₅—, —(CH₂)₂—O—(CH₂)₂— or —(CH₂)₂—S—(CH₂)₂—,
R²² represents hydrogen, —SO₂R¹¹, represents —COR¹¹ or —CO₂R¹¹; represents C₁-C₁₆-alkyl or C₂-C₁₆-alkenyl, each of which is optionally mono- or polysubstituted by identical or different substituents from the group consisting of fluorine, chlorine, bromine, cyano, methylamino, ethylamino, di-(C₁-C₆-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₁₀-cycloalkyl, C₃-C₆cycloalkyl-C₁-C₄-alkyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted with identical or different substituents from the group consisting of fluorine, chlorine, bromine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
w represents 0, 1 or 2,
n represents 1 or 2,
p represents 0 or 1.

5. Δ¹-Pyrrolines of the formula (I) according to claim 1, in which
R¹ represents fluorine, chlorine, bromine, methyl, trifluoromethyl, methoxy, trifluoromethoxy, methylthio or trifluoromethylthio,
R² and R³ independently of one another represent hydrogen, fluorine, chlorine, bromine, methyl, trifluoromethyl, methoxy or trifluoromethoxy,
R⁴ represents chlorine, bromine, methyl, ethyl, n-propyl, isopropyl, n-butyl, isobutyl, sec-butyl, tert-butyl, trifluoromethyl, trifluoroethyl or represents —SO₃R⁸,
R⁵ represents hydrogen, chlorine, bromine, methyl, ethyl, n-propyl, isopropyl, n-butyl, isobutyl, sec-butyl, tert-butyl, trifluoromethyl, trifluoroethyl or represents —SO₃R⁸,
R⁴ and R⁵ furthermore together represent the grouping 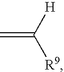
R⁶ represents fluorine, chlorine, methyl, ethyl, n-propyl, isopropyl, n-butyl, isobutyl, sec-butyl, tert-butyl, trifluoromethyl, trifluoroethyl, methoxy, ethoxy, n-propoxy, isopropoxy, n-butoxy, isobutoxy, sec-butoxy, tert-butoxy, trifluoromethoxy, trifluoroethoxy, methylthio, ethylthio, n-propylthio, isopropylthio, n-butylthio, isobutylthio, sec-butylthio, tert-butylthio, trifluoromethylthio or trifluoroethylthio,
m represents 0 or 1,
Q represents the grouping —X—Y-Z-E, with the proviso that Y does not represent a direct bond if X does not represent a direct bond,
R⁸ represents hydrogen, methyl, ethyl, n-propyl, isopropyl, trifluoromethyl or trifluoroethyl,
R⁹ represents hydrogen, methyl, ethyl, n-propyl, isopropyl, trifluoromethyl, trifluoroethyl, or represents phenyl, furyl, thienyl, pyrrolyl or pyridinyl, each of which is optionally mono- to trisubstituted by identical or different radicals from the list W¹,
X represents a direct bond, oxygen, sulphur, —SO₂—, —NR¹⁰—, —CO—, —C(O)—O—, —O—C(O)—, —CH₂—, —(CH₂)₂—, —C═C— (E or Z), —C≡C—, —CH₂O—, —(CH₂)₂O—, —OCH₂—, —O(CH₂)₂—, —O—CH₂—O—, —SCH₂—, —S(CH₂)₂—, —CH₂S— or —(CH₂)₂S—,
Y represents a direct bond or represents 1,4-phenylene, 1,3-phenylene, 2,6-naphthylene, 2,7-naphthylene, 2,4-furylene, 2,4-thienylene, 2,5-pyridinylene, 2,5-pyrimidinylene, 3,6-pyridazinylene or 2,5-pyrazinylene, each of which is optionally mono- or disubstituted by identical or different radicals from the list W¹,
Z represents a direct bond, methylene or ethylene,
E represents hydrogen, fluorine, chlorine, bromine, hydroxyl, cyano, formyl, —S(O)_{w}R¹¹, —OSO₂R¹¹, —NR¹²R¹³, —COR¹¹, —CO₂R¹¹, —OC(O)R¹¹, —CONR¹⁴R¹⁵, —N(R¹⁶)COR¹⁷, —SO₂NR²⁰R²¹; represents C₁-C₁₆-alkyl, C₂-C₁₆-alkenyl, C₁-C₁₆-alkoxy, C₂-C₁₆-alkenyloxy, each of which is optionally mono- or polysubstituted by identical or different radicals from the group consisting of fluorine, chlorine, bromine, cyano, C₁-C₆-alkoxy and —NR¹²R¹³; or represents cyclopropyl, cyclopentyl, cyclohexyl, cyclopropyloxy, cyclopentyloxy, cyclohexyloxy, cyclopropylmethyl, cyclopentylmethyl, cyclohexylmethyl, phenyl, phenoxy, benzyl, phenylethyl, benzyloxy, tetrazolyl, furyl, furfuryl, benzofuryl, tetrahydrofuryl, thienyl, thenyl, benzothienyl, thiolanyl, pyrrolyl, indolyl, pyrrolinyl, pyrrolidino, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, piperidino, morpholinyl, thiomorpholinyl, morpholino, thiomorpholino, triazinyl, triazolyl, quinolinyl or isoquinolinyl, each of which is optionally mono- to trisubstituted by identical or different radicals from the group consisting of fluorine, chlorine, bromine, cyano, methyl, ethyl, n-propyl, isopropyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, vinyl, allyl, 1-propenyl, butenyl, —CF═CHF, —CF═CH₂, —CF═CCl₂, —CH═CF₂, —CF₂CF═CF₂, —CH═CFH, —CH₂CF═CF₂, —CF═CF₂, —CF₂CH═CF₂, methoxy, ethoxy, n-propoxy, isopropoxy, n-butoxy, isobutoxy, sec-butoxy, tert-butoxy, trifluoromethoxy, difluoromethoxy, chlorodifluoromethoxy, trifluoroethoxy, methylthio, ethylthio, n-propylthio, isopropylthio, n-butylthio, isobutylthio, sec-butylthio, tert-butylthio, trifluoromethylthio, difluoromethylthio, chlorodifluoromethylthio, trifluoroethylthio,
W¹ represents fluorine, chlorine, bromine, cyano, formyl, methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy, tert-butoxy, vinyl, allyl, trifluoromethyl, trifluoroethyl, trifluoromethoxy, trifluoroethoxy, —OCF₂CF₂H, —CH═CF₂, —CH═CCl₂, —OCF═CF₂, —COMe, —COEt, —CO₂Me, —CO₂Et, —CO₂(t-Bu), —SMe, —SOMe, —SO₂Me, —SCF₃, —SOCF₃, —SO₂CF₃, —SCHF₂, —SOCHF₂, —SO₂CHF₂, —SO₂NMe₂, —NMe₂, —NEt₂, —N(n-Pr)₂, —N(Me)COMe, —N(Me)COEt, —N(Me)COPr, —N(Me)CO(t-Bu), 2-pyrrolidon-5-yl, 2-piperidon-6-yl, —N(Me)SO₂Me, —N(Me)SO₂Et, —N(Me)SO₂CF₃, —N(Et)SO₂CF₃, —N(Me)SO₂(CF₂)₃CF₃ or —OSO₂NMe₂,
R¹⁰ represents methyl, ethyl, n-propyl, isopropyl, n-butyl, isobutyl, sec-butyl, tert-butyl, trifluoromethyl, trifluoroethyl, cyclopropyl, cyclopropylmethyl, cyclopentyl, cyclopentylmethyl, cyclohexyl, cyclohexylmethyl, phenyl, benzyl or phenylethyl,
R¹¹ represents methyl, ethyl, n-propyl, isopropyl, n-butyl, isobutyl, sec-butyl, tert-butyl, pentyl, hexyl, —CF₃, —CHF₂, —CCl₃, —CCl₂F, dimethyl-aminomethyl, dimethylaminoethyl, diethylaminomethyl, diethylaminoethyl, cyclopropyl, cyclopentyl, cyclohexyl, phenyl or benzyl,
R₁₂ and R₁₃ represent hydrogen, —SO₂R¹¹, represent C₁-C₁₆-alkyl, C₂-C₁₆—alkenyl, each of which is optionally mono- or polysubstituted by identical or different substituents from the group consisting of fluorine, chlorine, bromine, C₁-C₄-alkylcarbonyl, C₁-C₄-alkylcarbonyloxy, C₁-C₄-alkylamino, di-(C₁-C₄-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₈-cycloalkyl, cyclopropylmethyl, cyclopentylmethyl, cyclohexylmethyl, cyclopropylmethyl, cyclopentylethyl, cyclohexylethyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, cyano, C₁-C₄-alkyl, C₁-C₄-halogenoalkyl having 1 to 9 fluorine, chlorine and/or bromine atoms, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms,
R¹² and R¹³ furthermore together represent C₂-C₈-alkenylene which is optionally mono- or polysubstituted by identical or different substituents from the group consisting of fluorine, chlorine, bromine, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms or represent C₃-C₈-alkylene, where the alkylene chain may in each case be interrupted by —O—, —S— or —NR¹⁸—, and where the radical is optionally mono- or polysubstituted in the alkylene moiety by identical or different substituents from the group consisting of fluorine, chlorine, cyano, methyl, ethyl, n-propyl, isopropyl, n-butyl, isobutyl, sec-butyl, tert-butyl, C₁-C₄-halogenoalkyl having 1 to 9 fluorine, chlorine and/or bromine atoms, methoxy, ethoxy, n-propoxy, isopropoxy, n-butoxy, isobutoxy, sec-butoxy, tert-butoxy, C₁-C₄-halogenoalkoxy having 1 to 9 fluorine, chlorine and/or bromine atoms, methylthio, ethylthio, n-propylthio, isopropylthio, n-butylthio, isobutylthio, sec-butylthio, tert-butylthio and C₁-C₄-halogenoalkylthio having 1 to 9 fluorine, chlorine and/or bromine atoms,
R¹⁴ and R¹⁵ independently of one another represent hydrogen, —SO₂CF₃, methyl, ethyl, n-propyl, isopropyl, n-butyl, isobutyl, sec-butyl, tert-butyl, pentyl, hexyl, —CF₃, —CH₂CF₃, —(CF₂)₃CF₃, cyclopropyl, cyclopentyl, cyclohexyl, methoxymethyl, methoxyethyl, or represent phenyl or benzyl, each of which is optionally mono- to trisubstituted by identical or different substituents from the group consisting of fluorine, chlorine, bromine, methyl, trifluoromethyl, methoxy, trifluoromethoxy,
R¹⁴ and R¹⁵ furthermore together represent —(CH₂)₄—, —(CH₂)₅—, —(CH₂)₆—, —CH₂—CH(CH₃)—CH₂—CH(CH₃)—CH₂—, —(CH₂)₂—O—(CH₂)₂—, —(CH₂)₂—S—(CH₂)₂— or —(CH₂)₂—N(R²²)—(CH₂)₂—,
R¹⁶ and R¹⁷ independently of one another represent methyl, ethyl, n-propyl, isopropyl, n-butyl, isobutyl, sec-butyl, tert-butyl, n-hexyl, trifluoromethyl, trifluoroethyl, cyclopropyl, cyclopentyl, cyclohexyl or represent phenyl or benzyl, each of which is optionally mono- to tetrasubstituted by identical or different substituents from the group consisting of fluorine, chlorine, bromine, methyl, trifluoromethyl, methoxy and trifluoromethoxy,
R¹⁶ and R¹⁷ furthermore together represent —(CH₂)₃—, —(CH₂)₄—, —(CH₂)₅—, —(CH₂)₆—, each of which is optionally mono- to tetrasubstituted by identical or different substituents from the group consisting of fluorine, chlorine, bromine, methyl, ethyl, methoxy, ethoxy, methylthio, ethylthio, trifluoromethyl, trifluoromethoxy and trifluoromethylthio,
R²⁰ and R²¹ independently of one another represent hydrogen, methyl, ethyl, n-propyl, isopropyl, n-butyl, isobutyl, sec-butyl, tert-butyl, n-hexyl, trifluoromethyl, trifluoroethyl, cyclopropyl, cyclopentyl or cyclohexyl,
R²⁰ and R²¹ furthermore together represent —(CH₂)₃—, —(CH₂)₄—, —(CH₂)₅—, —(CH₂)₂—O—(CH₂)₂— or —(CH₂)₂—S—(CH₂)₂—,
R²² represents hydrogen, —SO₂R¹¹, represents —COR¹¹ or —CO₂R¹¹; represents C₁-C₁₆-alkyl, C₂-C₁₆-alkenyl, each of which is optionally mono- or polysubstituted by identical or different substituents from the group consisting of fluorine, chlorine, bromine, cyano, methylamino, ethylamino, di-(C₁-C₆-alkyl)amino, C₁-C₄-alkoxy, C₁-C₄-halogenoalkoxy having 1 to 9 fluorine, chlorine and/or bromine atoms, C₁-C₄-alkylthio and C₁-C₄-halogenoalkylthio having 1 to 9 fluorine, chlorine and/or bromine atoms; represents C₃-C₈-cycloalkyl, cyclopropylmethyl, cyclopentylmethyl, cyclohexylmethyl, cyclopropylmethyl, cyclopentylethyl, cyclohexylethyl, phenyl, benzyl, phenylethyl, tetrazolyl, furyl, furfuryl, benzofuryl, tetrahydrofuryl, thienyl, thenyl, benzothienyl, thiolanyl, pyrrolyl, indolyl, pyrrolinyl, pyrrolidinyl, oxazolyl, benzoxazolyl, isoxazolyl, imidazolyl, pyrazolyl, thiazolyl, benzothiazolyl, thiazolidinyl, pyridinyl, pyrimidinyl, pyridazyl, pyrazinyl, piperidinyl, morpholinyl, thiomorpholinyl, triazinyl, triazolyl, quinolinyl or isoquinolinyl, each of which is optionally mono- to trisubstituted by identical or different substituents from the group consisting of fluorine, chlorine, bromine, cyano, methyl, ethyl, n-propyl, isopropyl, n-butyl, isobutyl, sec-butyl, tert-butyl, C₁-C₄-halogenoalkyl having 1 to 9 fluorine, chlorine and/or bromine atoms, methoxy, ethoxy, n-propoxy, isopropoxy, n-butoxy, isobutoxy, sec-butoxy, tert-butoxy, C₁-C₄-halogenoalkoxy having 1 to 9 fluorine, chlorine and/or bromine atoms, methylthio, ethylthio, n-propylthio, isopropylthio, n-butylthio, isobutylthio, sec-butylthio, tert-butylthio and C₁-C₄-halogenoalkylthio having 1 to 9 fluorine, chlorine and/or bromine atoms.

6. Δ¹-Pyrrolines of the formula (I-1) 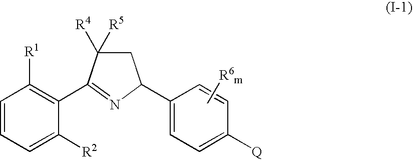
in which
R¹ represents fluorine or chlorine,
R² represents hydrogen or fluorine,
R⁴, R⁵, R⁶, m and Q have the meanings given in one of claims 1 to 5.

7. Δ¹-Pyrrolines of the formula (I-1) according to claim 6, in which R¹ and R² each represent fluorine.

8. Δ¹-Pyrrolines of the formula (I) according to any of claims 1 to 5, in which
X represents a direct bond,
Y represents phenylene, preferably 1,4-phenylene.

9. Δ¹-Pyrrolines of the formula (I) according to any of claims 1 to 5, in which E represents trifluoromethoxy.

10. Process for preparing compounds of the formula (I) according to claim 1, characterized in that
A) Δ¹-pyrrolines of the formula (I-a) 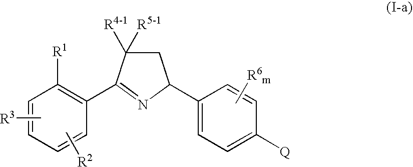
in which
R¹, R², R³, R⁶, m and Q have the meanings given in claim 1,
R⁴⁻¹ represents chlorine or bromine,
R⁵⁻¹ represents hydrogen, chlorine or bromine,
are obtained by reacting
A1) Δ¹-pyrrolines of the formula (II) 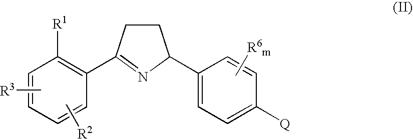
in which
R¹, R², R³, R⁶, m and Q having the meanings given in claim 1,
with a succinimide of the formula (III) 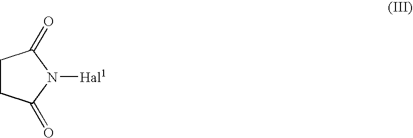
in which
Hal¹ represents chlorine or bromine,
or
A2) Δ¹-pyrrolines of the formula (II-a) 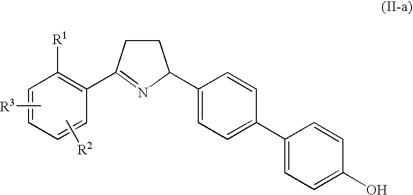
in which
R¹, R² and R³ have the meanings given in claim 1,
with a sulphonylhalide of the formula (IV) 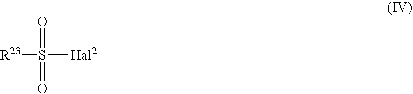
in which
Hal² represents chlorine or bromine,
R²³ represents alkyl or halogenoalkyl,
if appropriate in the presence of an acid binder and if appropriate in the presence of a diluent,
or
B) Δ¹-pyrrolines of the formula (I-b) 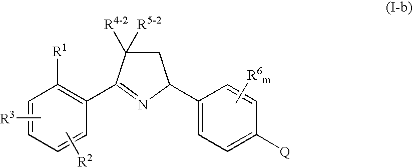
in which
R¹, R², R³, R⁶, m and Q have the meanings given in claim 1,
R⁴² represents alkyl or halogenoalkyl,
R⁵⁻² represents hydrogen, alkyl or halogenoalkyl,
are obtained by treating aminoketones of the formula (V) 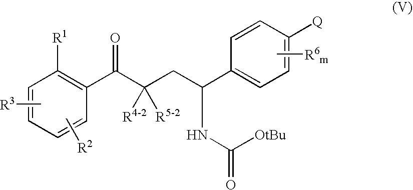
in which
R¹, R², R³, R⁶, m and Q have the meanings given in claim 1,
R⁴⁻² and R⁵⁻² have the meanings given above,
with a Lewis acid or a protonic acid, if appropriate in the presence of a diluent,
or
C) Δ¹-pyrrolines of the formula (I-c) 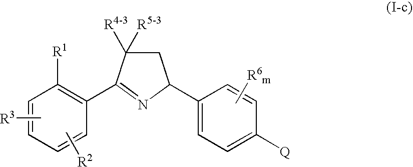
in which
R¹, R², R³, R⁶, m and Q have the meanings given in claim 1,
R⁴⁻³ represents —SO₃R⁸, where R⁸ has the meanings given in claim 1,
R⁵⁻³ represents hydrogen,
are obtained by reacting cyclopropanes of the formula (VI) 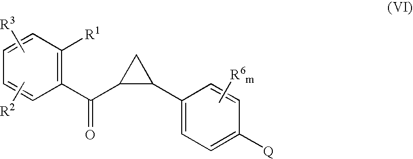
in which
R¹, R², R³, R⁶, m and Q have the meanings given in claim 1,
with sulphuric acid and acetonitrile and, if appropriate, in the presence of an alcohol of the formula (VII)
R⁸—OH (VII)
in which
R⁸ has the meanings given in claim 1,
or
D) Δ¹-pyrrolines of the formula (I-d) 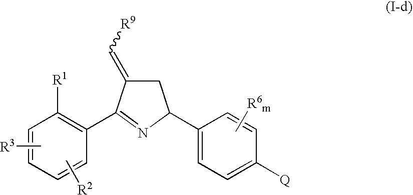
in which
R¹, R², R³, R⁶, m, Q and R⁹ have the meanings given in claim 1,
are obtained by reacting Δ¹-pyrrolines of the formula (II) 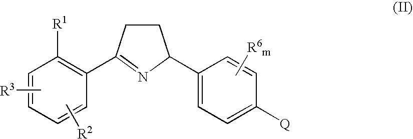
in which
R¹, R², R³, R⁶, m and Q have the meanings given in claim 1,
with an aldehyde of the formula (VIII)
R⁹—CHO (VIII)
in which
R⁹ has the meanings given in claim 1,
if appropriate in the presence of a base and if appropriate in the presence of a diluent.

11. Δ¹-Pyrrolines of the formula (I-e) 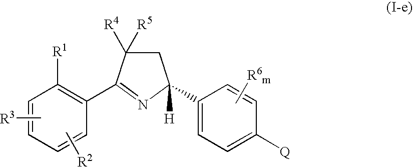
in which
R¹, R², R³, R⁴, R⁵, R⁶, m and Q have the meanings given in claim 1.

12. Δ¹-Pyrrolines of the formula (I-f) 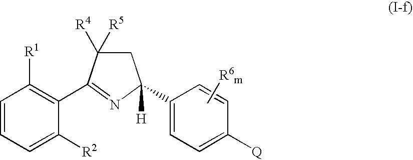
in which
R¹ represents fluorine or chlorine,
R² represents hydrogen or fluorine and
R³, R⁴, R⁵, R⁶, m and Q have the meanings given in claim 1.

13. Aminoketones of the formula (V) 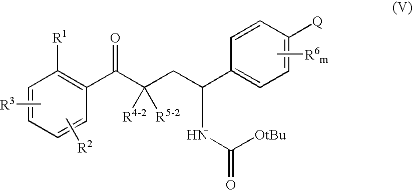
in which
R¹, R², R³, R⁶, m and Q have the meanings given in claim 1,
R²⁻⁴ represents alkyl or halogenoalkyl,
R⁵⁻² represents hydrogen, alkyl or halogenoalkyl.

14. Lactams of the formula (IX) 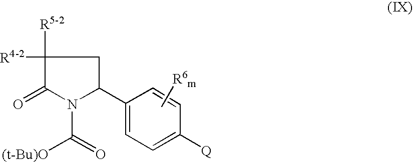
in which
R⁶, m and Q have the meanings given in claim 1,
R⁴⁻² represents alkyl or halogenoalkyl,
R⁵⁻² represents hydrogen, alkyl or halogenoalkyl.

15. Pesticides, characterized in that they comprise at least one compound of the formula (I) according to claim 1, in addition to extenders and/or surfactants.

16. Use of compounds of the formula (I) according to claim 1 for controlling pests.

17. Method for controlling pests, characterized in that compounds of the formula (I) according to claim 1 are allowed to act on pests and/or their habitat.

18. Process for preparing pesticides, characterized in that compounds of the formula (I) according to claim 1 are mixed with extenders and/or surfactants.
